(19) United States
(12) Patent Application Publication
(10) Pub. No.: US 2022/0218851 A1
(43) Pub. Date: Jul. 14, 2022

(54) COMPOUNDS AND METHODS FOR IMAGING IMMUNE ACTIVITY

(71) Applicant: Board of Regents, The University of Texas System, Austin, TX (US)

(72) Inventors: David PIWNICA-WORMS, Houston, TX (US); Federica PISANESCHI, Houston, TX (US); Seth GAMMON, Houston, TX (US)

(73) Assignee: Board of Regents, The University of Texas System, Austin, TX (US)

(21) Appl. No.: 17/604,305

(22) PCT Filed: Apr. 16, 2020
(86) PCT No.: PCT/US2020/028438

§ 371 Date: Oct. 15, 2021

Related U.S. Application Data
(60) Provisional application No. 62/834,779, filed on Apr. 16, 2019.

(51) Int. Cl. A61K 51/04

(52) U.S. Cl. CPC A61K 51/0459

(57) ABSTRACT

The present disclosure provides radiolabeled compounds of the formula: (I) and (II), as well as precursor compounds of the formula: (VII) wherein the variables are defined herein. The present disclosure also provides radiopharmaceutical compositions comprising the radiolabeled compounds disclosed herein as well as precursor compositions comprising the precursor compounds disclosed herein. The present disclosure further provides methods of imaging using the radiolabeled compounds and/or radiopharmaceutical compositions of the present disclosure as well as kits for the preparation of the radiolabeled compounds and radiopharmaceutical compositions disclosed herein.

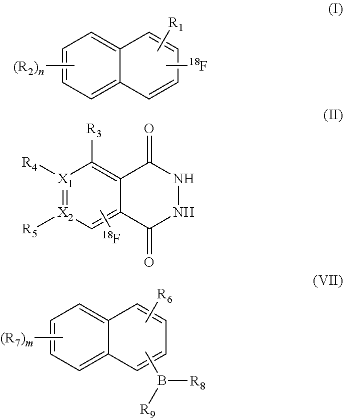

This application claims the benefit of priority to U.S. Provisional Application No. 62/834,779, filed on Apr. 16, 2019, the entire contents of which are hereby incorporated by reference.

### BACKGROUND

### I. Field

The present disclosure relates to the fields of radiopharmaceuticals, imaging, and diagnostics. More specifically, it relates to compounds and compositions thereof which are useful, for example, as radiotracers in positron-emission tomography.

### II. Description of Related Art

Dysregulation of the innate immune system contributes to the pathology of a variety of diseases that impact millions of people. For cancer there is great complexity. The tumor microenvironment (TME) plays an important role in immune inhibition, immunosurveillance, and immuno-editing (Bilusic and Gulley, 2017). Besides cancer and mesenchymal cells, the TME contains a variety of immune cells, including myeloid-derived suppressor cells, regulatory T cells, tumor-associated macrophages, helper and effector cytotoxic T cells, dendritic cells, and several pro- and anti-inflammatory cytokines secreted by both cancer and immune cells. Innate immunity, through the action of neutrophils, can suppress tumors, but depending on context, neutrophils and granulocytic myeloid-derived suppressor cells (GrMDSC) can also promote tumor growth (Rakic et al., 2018, Aarts and Kuijpers, 2018, Rymaszewski et al., 2014) and, in preclinical models, block immunotherapy. Preclinical and clinical studies have evaluated the ability of immunotherapeutic agents to cause local inflammation, improve tumor recognition, and generate an immune response against a broad spectrum of antigens. Agents studied have included immunomodulatory antibodies (CD40, CD137, OX40, GITR), immune checkpoint therapies (anti-CTL-4, anti-PD1, anti-PDL1), cytokines (IL-2, IL-12, GM-CSF), TLR agonists, STING agonists, cancer vaccines, and oncolytic viruses (Bilusic and Gulley, 2017). In cancer immunotherapy, a main challenge is to generate T cell responses in patients with immunologically “cold” tumors. Preclinical and early clinical studies have suggested that intratumoral therapies (in situ vaccination or localized radiation), alone or in combination with systemic immunotherapy, are able to convert a “cold” tumor to a “hot” tumor, thereby increasing the potential for a response to immune checkpoint blockade (Bilusic and Gulley, 2017). A uniform functional designation of a “hot” tumor remains to be defined.

On other fronts, obesity and diabetes affect the global inflammatory state of the body leading to a variety of complications (Stone et al., 2018), including increasing risk for tumor development (Park et al., 2018, Anastasi et al., 2018, Sfanos et al., 2017, and Corrêa et al., 2017). Atherosclerotic cardiovascular disease (CVD) is the leading cause of morbidity, mortality, and health care costs in the developed world, a distinction that is projected to apply globally within the next decade (Wagner and Brath, 2012 and Go et al., 2013). Many metabolic and hemodynamic factors influence atherosclerosis progression, defined by arterial wall inflammation (Hansson, 2005). Atherosclerosis often first presents as a major adverse cardiovascular event (MACE), suggesting that identifying high-risk patients with subclinical disease before the first MACE is a vital prevention strategy (Naghavi et al., 2003). Many proposed biomarkers for risk stratification target the inflammation underlying plaque development and instability (McDonnell et al., 2009). Furthermore auto-immune inflammatory diseases such as rheumatoid arthritis, colitis, and lupus all contribute to significant morbidity and mortality (Li et al., 2018, Skopelja-Gardner et al., 2018, and Morell et al., 2017). Finally, there appears to be a Janus role for innate immunity in the pathophysiology of MS, nephritis, viral infection, and wound healing where in early stages the innate immune system is destructive (Wojkowska et al., 2014 and Pierson et al., 2018), while later the innate immune system may indeed help control the total inflammatory state through inhibition of T-cells and dendritic cells (Leliefeld et al., 2015 and Mayadas et al., 2010).

Many of these diseases and the inflammatory states occur at deep tissue sites or sites that may not be amenable for repetitive biopsy, limiting access to vital information to inform therapeutic choices. Furthermore, peripheral assessment of cytokines or other blood biomarkers may not be indicative of the local tumor microenvironment and thus, the need remains for radiotracers and methods that enable local assessment in vivo in patients.

### SUMMARY

In some aspects, the present disclosure provides compounds of the formula:

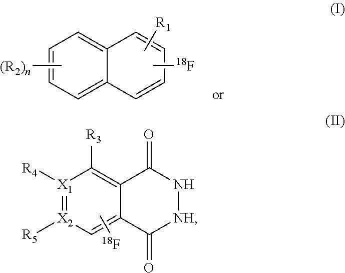

wherein:
n is 0-6;
R₁ is —ORₐ or —NR_{b}R_{c}, wherein:
   Rₐ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{b} and R_{c} are each independently hydrogen or a monovalent amine protecting group; or
      alkyl_{(C≤12)}, aralkyl_{(C≤12)}, acyl_{(C≤12)}, or a substituted version of any of these groups; or
   R_{b} and R_{c} are taken together and are a divalent amine protecting group;
R₂, in each instance, is independently hydrogen, hydroxy, halo, amino, nitro, carboxy, or mercapto; or
   —Y—R_{d}, wherein:
      Y is a covalent bond, —C(O)—, —OC(O)—, —NHC(O)—;
      R_{d} is alkyl_{(C≤12)}, alkoxy_{(C≤12)}, alkylamino_{(C≤12)}, dialkylamino_{(C≤12)}, or a substituted version of any of these groups;
R₃ is hydrogen, —ORₑ or —NR_{f}R_{g}, wherein:
   Rₑ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{f} and R_{g} are each independently hydrogen or a monovalent amine protecting group; or
      alkyl_{(C≤12)}, aralkyl_{(C≤12)}, acyl_{(C≤12)}, or a substituted version of any of these groups; or
   R_{f} and R_{g} are taken together and are a divalent amine protecting group;
R₄ and R₅ are each independently absent, hydrogen, hydroxy, amino, cyano, nitro, or halo; or
   alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
X₁ and X₂ are each independently —C═ or —N═;
   or a pharmaceutically acceptable salt of either of these formulae.

In some embodiments, the compounds are further defined as:

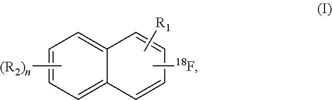

wherein:
n is 0-6;
R₁ is —ORₐ or —NR_{b}R_{c}, wherein:
   Rₐ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{b} and R_{c} are each independently hydrogen or a monovalent amine protecting group; or
      alkyl_{(C≤12)}, aralkyl_{(C≤12)}, acyl_{(C≤12)}, or a substituted version of any of these groups; or
   R_{b} and R_{c} are taken together and are a divalent amine protecting group;
R₂, in each instance, is independently hydrogen, hydroxy, halo, amino, nitro, carboxy, or mercapto; or
   —Y—R_{d}, wherein:
      Y is a covalent bond, —C(O)—, —OC(O)—, —NHC(O)—;
      R_{d} is alkyl_{(C≤12)}, alkoxy_{(C≤12)}, alkylamino_{(C≤12)}, dialkylamino_{(C≤12)}, or a substituted version of any of these groups;
         or a pharmaceutically acceptable salt thereof.

In some embodiments, the compounds are further defined as:

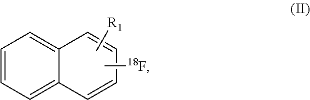

wherein:
R₁ is —ORₐ or —NR_{b}R_{c}, wherein:
   Rₐ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{b} and R_{c} are each independently hydrogen or a monovalent amine protecting group; or
      alkyl_{(C≤12)}, aralkyl_{(C≤12)}, acyl_{(C≤12)}, or a substituted version of any of these groups; or
   R_{b} and R_{c} are taken together and are a divalent amine protecting group;
      or a pharmaceutically acceptable salt thereof.

In some embodiments, the compounds are further defined as:

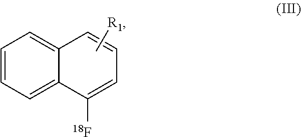

wherein:
R₁ is —ORₐ or —NR_{b}R_{c}, wherein:
   Rₐ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{b} and R_{c} are each independently hydrogen or a monovalent amine protecting group; or
      alkyl_{(C≤12)}, aralkyl_{(C≤12)}, acyl_{(C≤12)}, or a substituted version of any of these groups; or
   R_{b} and R_{c} are taken together and are a divalent amine protecting group;
      or a pharmaceutically acceptable salt thereof.

In some embodiments, the compounds are further defined as:

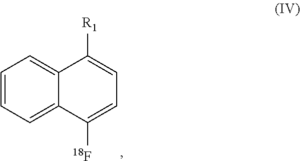

wherein:
R₁ is —ORₐ or —NR_{b}R_{c}, wherein:
   Rₐ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{b} and R_{c} are each independently hydrogen or a monovalent amine protecting group; or
      alkyl_{(C≤12)}, aralkyl_{(C≤12)}, acyl_{(C≤12)}, or a substituted version of any of these groups; or
   R_{b} and R_{c} are taken together and are a divalent amine protecting group;
      or a pharmaceutically acceptable salt thereof.

In some embodiments, Rₐ is hydrogen, —S(O)₂OH, —C(O)-alkoxy_{(C≤12)}, substituted —C(O)-alkoxy_{(C≤12)}, heterocycloalkyl_{(C≤12)}, or substituted heterocycloalkyl_{(C≤12)}. In some embodiments, Rₐ is hydrogen. In other embodiments, Rₐ is —S(O)₂OH. In still other embodiments, Rₐ is —C(O)-alkoxy_{(C≤12)} or substituted —C(O)-alkoxy_{(C≤12)}. In further embodiments, Rₐ is —C(O)-alkoxy_{(C≤12)}, such as —C(O)—OtBu. In yet other embodiments, Rₐ is heterocycloalkyl_{(C≤12)} or substituted heterocycloalkyl_{(C≤12)}. In further embodiments, Rₐ is substituted heterocycloalkyl_{(C≤12)}, such as 2-carboxy-3,4,5-trihydroxytetrahydro-2H-pyran-6-yl.

In some embodiments, the compounds are further defined as:

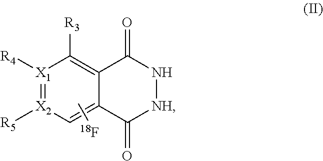

R₃ is hydrogen, —ORₑ or —NR_{f}R_{g}, wherein:
   Rₑ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{f} and R_{g} are each independently hydrogen or a monovalent amine protecting group; or
      alkyl_{(C≤12)}, aralkyl_{(C≤12)}, acyl_{(C≤12)}, or a substituted version of any of these groups; or
   R_{f} and R_{g} are taken together and are a divalent amine protecting group;
R₄ and R₅ are each independently absent, hydrogen, hydroxy, amino, cyano, nitro, or halo; or
   alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and X₁ and X₂ are each independently —C═ or —N═;
or a pharmaceutically acceptable salt thereof.

In some embodiments, the compounds are further defined as:

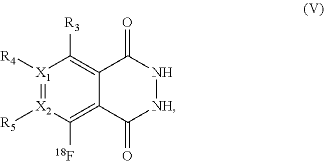

wherein:
R₃ is hydrogen, —ORₑ or —NR_{f}R_{g}, wherein:
   Rₑ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{f} and R_{g} are each independently hydrogen or a monovalent amine protecting group; or
      alkyl_{(C≤12)}, aralkyl_{(C≤12)}, acyl_{(C≤12)}, or a substituted version of any of these groups; or
   R_{f} and R_{g} are taken together and are a divalent amine protecting group;
R₄ and R₅ are each independently absent, hydrogen, hydroxy, amino, cyano, nitro, or halo; or
   alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
X₁ and X₂ are each independently —C═ or —N═;
   or a pharmaceutically acceptable salt thereof.

In some embodiments, the compounds are further defined as:

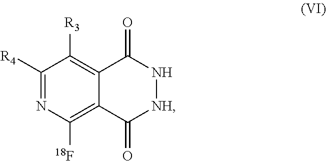

wherein:
R₃ is hydrogen, —ORₑ or —NR_{f}R_{g}, wherein:
   Rₑ is hydrogen, —S(O)₂OH, or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   R_{f} and R_{g} are each independently hydrogen or a monovalent amine protecting group; or
      alkyl_{(C≤12)}, aralkyl_{(C≤12)}, acyl_{(C≤12)}, or a substituted version of any of these groups; or
   R_{f} and R_{g} are taken together and are a divalent amine protecting group;
R₄ is hydrogen, hydroxy, amino, cyano, nitro, or halo; or
   alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
      or a pharmaceutically acceptable salt thereof.

In some embodiments, R₄ is aryl_{(C≤12)} or substituted aryl_{(C≤12)}. In further embodiments, R₄ is aryl_{(C≤12)}, such as phenyl. In some embodiments, R_{f} is hydrogen. In some embodiments, R_{g} is hydrogen.

In some embodiments, the compound is further defined as:

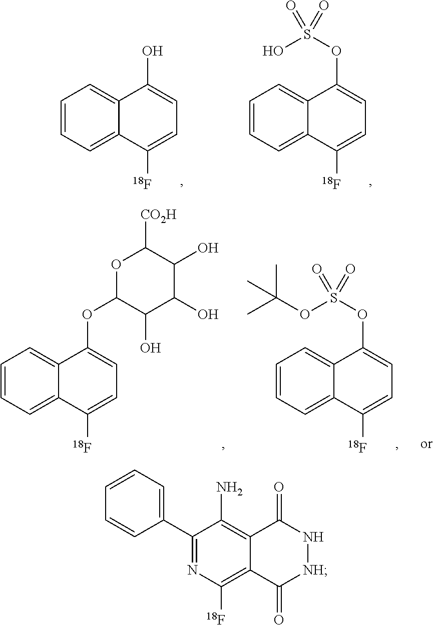

or a pharmaceutically acceptable salt thereof.

In another aspect, the present disclosure provides radiopharmaceutical compositions comprising:

(a) a radiolabeled compound of the present disclosure; and
(b) a pharmaceutically acceptable carrier.

In some embodiments, the composition is formulated for administration: intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, parenterally, subconjunctival, subcutaneously, via injection, via local delivery, or via localized perfusion. In further embodiments, the composition is formulated for administration intravenously or via injection. In some embodiments, the composition is formulated as a unit dose.

In yet another aspect, the present disclosure provides methods of imaging a subject comprising:
(a) administering to the subject an effective amount of a radiolabeled compound or radiopharmaceutical composition of the present disclosure; and
(b) obtaining at least one image of a portion of the subject.

In some embodiments, the subject is a vertebrate. In further embodiments, the vertebrate is a mammal, such as a human. In some embodiments, the at least one image is a positron-emission tomography image. In some embodiments, the methods are suitable for detecting and/or measuring one or more biomarkers associated with inflammation. In some embodiments, the methods are suitable for detecting and/or measuring the activation of a biochemical pathway associated with inflammation. In some embodiments, the inflammation is caused by or results in an ROS and/or an RNS. In further embodiments, the ROS and/or the RNS are produced by a Fenton reaction. In some embodiments, the methods further comprise detecting a level of activity of an enzyme. In some embodiments, the enzyme is a peroxidase, such as myeloperoxidase. In other embodiments, the enzyme is an NADPH oxidase, such as NOX1, NOX2, NOX3, or NOX4. In still other embodiments, the enzyme is a nitric oxide synthase, such as iNOS, nNOS, or eNOS. In other embodiments, the enzyme is a xanthine oxidase or dual oxidase peroxidase.

In some embodiments, the method further comprises diagnosing, prognosing, staging, or monitoring the progression of a disease or disorder. In some embodiments, the disease or disorder is a cardiovascular disease, cancer, a neurological disorder, an autoimmune disease, obesity or a condition associated with obesity, a condition associated with radiation, a bacterial infection, a viral infection, a parasitic infection, inflammation or a condition associated with inflammation. In some embodiments, the disease or disorder is inflammation or a condition associated with inflammation, such as pancreatitis, hepatitis, pneumonitis, adult respiratory distress syndrome, pulmonary fibrosis, cystic fibrosis, chronic obstructive pulmonary disease, asthma, dermatitis, gastritis, esophagitis, encephalitis, dementias, irritable bowel syndrome, inflammatory bowel disease, nephritis, muscle wasting, osteoarthritis, type 2 diabetes or a complication of type 1 or type 2 diabetes. In other embodiments, the disease or disorder is a neurological disorder. In further embodiments, the disease or disorder is a central neurologic disease, such as white matter inflammation, meningitis, vasculitis, autoimmune encephalitis, metabolic encephalitis, Alzheimer's Disease, and other dementias and degenerative inflammatory diseases of the brain. In other embodiments, the disease or disorder is a condition associated with radiation, such as post-radiation inflammation or fibrosis. In other embodiments, the disease or disorder is a cardiovascular disease, such as vasculitis, atherosclerosis, myocardial infarction, myocarditis, heart failure, pulmonary hypertension, or stroke. In still other embodiments, the disease or disorder is cancer, such as breast cancer, liver cancer, lung cancer, thyroid cancer, head and neck cancer, pancreatic cancer, colorectal cancer, prostate cancer, renal cancer, skin cancer, brain cancer, sarcoma, multiple myeloma, lymphoma, or leukemia. In some embodiments, the breast cancer is inflammatory breast cancer. In some embodiments, the breast cancer is triple-negative breast cancer. In other embodiments, the cancer is skin cancer, such as melanoma. In still other embodiments, the cancer is brain cancer, such as glioblastoma. In yet other embodiments, the disease or disorder is an autoimmune disease, such as psoriasis, multiple sclerosis, scleroderma, rheumatoid arthritis, lupus, psoriatic arthritis, ankylosing spondylitis, Sjögren syndrome, vitiligo, uveitis, dry eye syndrome, systemic sclerosis, type 1 diabetes, encephalitis, myasthenia gravis, or inflammatory bowel disease. In other embodiments, the disease or disorder is a bacterial infection, a viral infection, or a parasitic infection. In some embodiments, the disease or disorder is inflammation associated with a vector-borne disease. In some embodiments, the administering is via injection. In some embodiments, the method further comprises monitoring the progression of tissue repair.

In yet another aspect, the present disclosure provides precursor compounds of the formula:

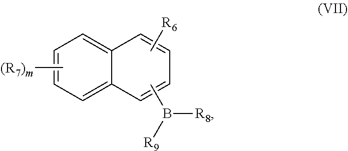

wherein:
m is 0-6;
R₆ is —ORₕ or —NRᵢRⱼ, wherein:
   Rₕ is —S(O)₂OH or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   Rᵢ and Rⱼ are each independently hydrogen or a monovalent amine protecting group; or
      alkyl_{(C≤12)}, aralkyl_{(C≤12)}, acyl_{(C≤12)}, or a substituted version of any of these groups; or
   Rᵢ and Rⱼ are taken together and are a divalent amine protecting group;
R₇, in each instance, is independently hydroxy, halo, amino, nitro, carboxy, or mercapto; or
   —Y—Rₖ, wherein:
      Y is a covalent bond, —C(O)—, —OC(O)—, —NHC(O)—;
      Rₖ is alkyl_{(C≤12)}, alkoxy_{(C≤12)}, alkylamino_{(C≤12)}, dialkylamino_{(C≤12)}, or a substituted version of any of these groups; and
R₈ and R₉ are each independently halo or hydroxy; or
   alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
   X₃ is alkanediyl_{(C≤12)}, substituted alkanediyl_{(C≤12)}, or a boronic acid protecting group;
      or a pharmaceutically acceptable salt thereof.

In some embodiments, the compounds are further defined as:

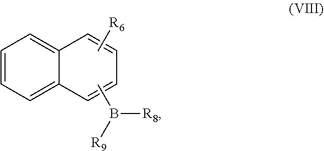

wherein:
R₆ is —ORₕ or —NRᵢRⱼ, wherein:
   Rₕ is —S(O)₂OH or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   Rᵢ and Rⱼ are each independently hydrogen or a monovalent amine protecting group; or
      alkyl_{(C≤12)}, aralkyl_{(C≤12)}, acyl_{(C≤12)}, or a substituted version of any of these groups; or
   Rᵢ and Rⱼ are taken together and are a divalent amine protecting group; and
R₈ and R₉ are each independently halo or hydroxy; or
   alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
   X₃ is alkanediyl_{(C≤12)}, substituted alkanediyl_{(C≤12)}, or a boronic acid protecting group;
      or a pharmaceutically acceptable salt thereof.

In some embodiments, the compounds are further defined as:

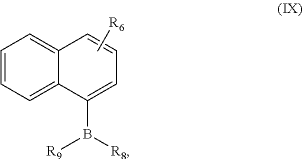

wherein:
R₆ is —ORₕ or —NRᵢRⱼ, wherein:
   Rₕ is —S(O)₂OH or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   Rᵢ and Rⱼ are each independently hydrogen or a monovalent amine protecting group; or
      alkyl_{(C≤12)}, aralkyl_{(C≤12)}, acyl_{(C≤12)}, or a substituted version of any of these groups; or
   Rᵢ and Rⱼ are taken together and are a divalent amine protecting group; and
R₈ and R₉ are each independently halo or hydroxy; or
   alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
   X₃ is alkanediyl_{(C≤12)}, substituted alkanediyl_{(C≤12)}, or a boronic acid protecting group;
      or a pharmaceutically acceptable salt thereof.

In some embodiments, the compounds are further defined as:

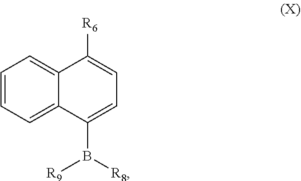

wherein:
R₆ is —ORₕ or —NRᵢRⱼ, wherein:
   Rₕ is —S(O)₂OH or a hydroxyl protecting group; or
      alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
   Rᵢ and Rⱼ are each independently hydrogen or a monovalent amine protecting group; or
      alkyl_{(C≤12)}, aralkyl_{(C≤12)}, acyl_{(C≤12)}, or a substituted version of any of these groups; or
   Rᵢ and Rⱼ are taken together and are a divalent amine protecting group; and
R₈ and R₉ are each independently halo or hydroxy; or
   alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
   X₃ is alkanediyl_{(C≤12)}, substituted alkanediyl_{(C≤12)}, or a boronic acid protecting group;
      or a pharmaceutically acceptable salt thereof.

In some embodiments, Rₕ is a hydroxyl protecting group. In other embodiments, Rₕ is heterocycloalkyl_{(C≤12)} or substituted heterocycloalkyl_{(C≤12)}. In further embodiments, Rₕ is substituted heterocycloalkyl_{(C≤12)}, such as 2-carboxy-3,4,5-trihydroxytetrahydro-2H-pyran-6-yl. In still other embodiments, Rₕ is —C(O)-alkoxy_{(C≤12)}, such as —C(O)—OtBu. In some embodiments, R₈ is hydroxy. In other embodiments, R_{g} is alkoxy_{(C≤12)}. In some embodiments, R₉ is hydroxy. In other embodiments, R₉ is alkoxy_{(C≤12)}. In some embodiments, R₈ and R₉ are taken together and are alkanediyl_{(C≤12)}, such as 1,1,2,2-tetramethylethanediyl. In some embodiments, the compound is further defined as:

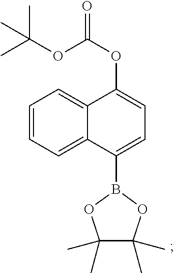 [SMILES: CC(C)(C)OC(=O)Oc1ccc(B2OC(C)(C)C(C)(C)O2)c2ccccc12]

or a pharmaceutically acceptable salt thereof.

In another aspect, the present disclosure provides precursor compositions comprising:

(a) a precursor compound of the present disclosure; and
(b) a pharmaceutically acceptable carrier.

In still another aspect, the present disclosure provides radiopharmaceutical kits for the preparation of a radiolabeled compound of the present disclosure, wherein the kit comprises a precursor compound or composition of the present disclosure. In some embodiments, the kit comprises or consists of a cassette. In some embodiments, the kit further comprises a pharmaceutically acceptable carrier. In some embodiments, the kit further comprises instructions for the preparation of the radiolabeled compound.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the general TracerLab scheme. The radiosynthesis was performed on a TracerLab FX (General Electric Healthcare, Münster, Germany) automatic module.

FIG. 2 shows HPLC trace of purified [¹⁸F]FN. Top: gamma channel; Bottom: UV channel (254 nm).

FIG. 3 shows HPLC trace of purified [¹⁸F]FNS. Top: gamma channel; Bottom: UV channel (254 nm).

FIG. 4 shows HPLC trace of purified [¹⁸F]FNG. Top: gamma channel; Bottom: UV channel (254 nm).

FIGS. 5A-5E show that [¹⁸F]4FN local retention depends on acute inflammation and may depend significantly on reactive oxygen or nitrogen species, ROS or RNS, respectively. Balb/c mice were treated with either PMA or vehicle and imaged 24 hr post treatment by PET/CT scan at 1 hr post-injection (IP) of [¹⁸F]4FN (FIG. 1A); photograph 3 hr post [¹⁸F]4FN (FIG. 1i). BLI 10 min post luminol injection (FIG. 1C). Aged-matched, 15-week-old female wild-type B16 or Nox2 KO mice (n=3 each) were treated with PMA or vehicle and imaged with [¹⁸F]4FN one-hour post IP administration of radiotracer. PET/CT data were quantified using ROIs surrounding the right and left ears. Contrast-to-noise ratios were calculated for all mice and data were plotted as mean and SEM. Nox2 KO mice had decreased contrast between PMA-treated and untreated ears, indicating a reactive oxygen species-dependent trapping in models of activation of the innate immune system (FIG. 1D). Radioactive HPLC trace and chemical structure of [¹⁸F]4FN (FIG. 1E).

FIGS. 6A-6D show VOI analysis of [¹⁸F]4FN in vivo in PMA-induced ear inflammation model. (FIGS. 6A & 6B) IP or IV delivery of imaging agent both yielded statistically detectable differences in SUVaverage values. (FIG. 6C) [¹⁸F]4FN can readily detect inflammation vs mock in vivo with large effect sizes. (FIG. 6D) Overall evaluation of IP and IV administration of [¹⁸F]4FN.

FIG. 7 multipanels show PET images of the 24 hr PMA ear model of inflammation. [¹⁸F]4FNS or [¹⁸F]FN were synthesized, injected into mice IV (n>=3) and imaged at 1 hr post tracer injection. The right ear was observably inflamed (left images) and readily detectable with both [¹⁸F]4FNS (right images) and [¹⁸F]FN (second from right images). A separate cohort of mice (>=3) were injected IV with [¹⁸F]FDG (second from left images), and while ears were visibly inflamed at this level, no FDG image asymmetry was observed.

FIG. 8 shows [¹⁸F]FNG PET imaging 1 h post injection. Left panel: comparison between PMA-inflamed ear and vehicle-treated ear. Right panel: PET MIP of mouse head.

FIG. 9 shows [¹⁸F]4FNS PET images of a 4T1 tumor (transaxial view; red oval), known to be heavily infiltrated with neutrophils and MDSCs, and easily distinguishable from the contralateral flank/leg that bears no tumor.

FIG. 10 demonstrates [¹⁸F]4FN PET analysis of an LPS-induced model of arthritis. Herein, C57BL6/N mice were injected i.a. with LPS, left ankle, or vehicle, right ankle. After 24 hours, mild inflammation of the left ankle was confirmed by visual inspection. 1 hour dynamic PET scans were acquired post i.v. injection of [¹⁸F]4FN. Mice were subsequently imaged for ROS by BLI (L-012). A) 1 hour static [¹⁸F]4FN PET image (left) and BLI (right). B) PET image-derived time-activity curves (TACs) were quantified for the ankles (left, n=4). Selective accumulation of [¹⁸F]4FN in inflamed ankles at 1 hr directly correlated on a per animal basis with the bioluminescence readout of inflammatory ROS bursts (L-012) (right). C) H&E staining cross-validated local inflammation (vehicle, left panel; LPS, right panel), utilizing the same mouse as imaged in A. D) Image-based biodistribution was calculated from the dynamic PET/CT data and SUV plotted for various organs over time.

FIG. 11 shows that ROS retention in neutrophil-like cells is inducible by PMA, a known inducer of inflammatory ROS burst, and can be blocked by known inhibitors of Nox2 (DPI) or MPO (4-ABAH). HL-60 cells were first differentiated to yield “neutrophil-like” cells. Then, in parallel, cell aliquots from each batch were imaged with L-012 (to confirm ROS production by bioluminescence) and incubated with 6.2 kBq of [¹⁸F]4FN. To induce ROS, all cells were incubated with PMA. To test dependence of trapping on ROS-producing enzymes, some cells were pre-incubated with Nox2 inhibitor (DPI) or MPO inhibitor (4-ABAH) prior to PMA. Data were quantified as a ratio of the cell-associated radioactive counts per milligram protein over concentration of radiotracer in the extracellular space, and then normalized to vehicle.

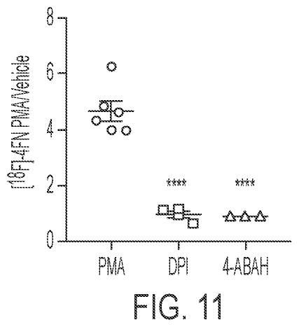

### DESCRIPTION OF ILLUSTRATIVE EMBODIMENTS

The present disclosure provides inter alia precursor compounds for ¹⁸F labeling, ¹⁸F-labeled compounds, and compositions thereof for use in medical imaging, such as positron-emission tomography (PET).

### I. RADIOLABELED COMPOUNDS OF THE PRESENT DISCLOSURE AND PRECURSORS THEREOF

| Compound Identifier | Structure |
|---|---|
| 4FN precursor (i.e., 2) | 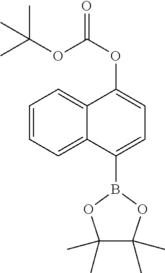 [SMILES: CC(C)(C)OC(=O)Oc1ccc(B2OC(C)(C)C(C)(C)O2)c2ccccc12] |
| [¹⁸F]4FN (i.e., 4) | 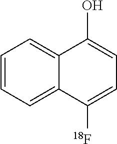 [SMILES: Oc1ccc([18F])c2ccccc12] |
| [¹⁸F]4FNS | 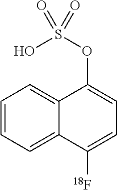 [SMILES: O=S(=O)(O)Oc1ccc([18F])c2ccccc12] |
| [¹⁸F]4FNG | 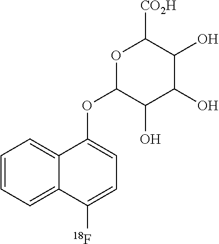 [SMILES: O=C(O)C1OC(Oc2ccc([18F])c3ccccc23)C(O)C(O)C1O] |
| [¹⁸F]F-L012 | 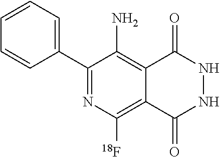 [SMILES: Nc1c(-c2ccccc2)nc([18F])c2c(=O)[nH][nH]c(=O)c12] |

The radiolabeled compounds of the present disclosure are shown, for example, above, in the summary section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present disclosure may in some embodiments be used for imaging, such as PET imaging. Imaging may be used to diagnose, prognose, stage, and/or monitor the progression of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the radiolabeled compounds of the present disclosure are deemed “active compounds” and “radiopharmaceuticals” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

The term “radiopharmaceutical” refers to a radiolabeled pharmaceutical or compound in a form suitable for administration to the mammalian, especially human, body. Radiopharmaceuticals may be used for diagnostic imaging or radiotherapy. The radiopharmaceuticals of the present disclosure are preferably used for diagnostic imaging.

In some embodiments, the compounds of the present disclosure have the advantage that they may be more efficacious than, be less toxic than, be longer acting than, be more selective than, produce fewer side effects than, be more easily absorbed than, more metabolically stable than, more lipophilic than, more hydrophilic than, and/or have a better pharmacokinetic profile (e.g., lower clearance) than, and/or have other useful pharmacological, radiopharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, unless specifically indicated, atoms making up the compounds of the present disclosure are intended to include all isotopic forms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### II. IMAGING

In some aspects, the present disclosure provides methods of imaging using the compounds and compositions of the present disclosure. In some embodiments, the imaging is positron-emission tomography. Positron emission tomography (PET) imaging is based on detecting two time-coincident high-energy photons from the emission of a positron-emitting radioisotope. PET imaging is unique in its very high sensitivity and accurate estimation of the in vivo concentration of the radiotracer. PET imaging has been widely adopted as an important clinical modality for oncological, cardiovascular, and neurological applications. PET imaging has also become an important tool in preclinical studies, particularly for investigating murine models of disease and other small-animal models.

In some aspects, the present disclosure provides compounds comprising a radioisotope, such as ¹⁸F. One of skill in the art will appreciate that other radioisotopes may also be used in placed of ¹⁸F in the composed disclosed herein. These other radioisotopes include, but are not limited to, ¹³¹I or ⁷⁶Br. Other isotopic species for labeling the present compounds may include ¹¹C and ¹³C.

### III. INFLAMMATION AND REACTIVE OXYGEN/NITROGEN SPECIES ACTIVITY

Molecular imaging approaches have the advantage broadly of allowing non-invasive and repetitive analysis of the biochemical status of local inflammatory environments, particularly in the context of tumors. However, deep tissue imaging techniques such as standard contrast MRI or 18-FDG PET lack the sensitivity or specificity to detect changes in the pathophysiologic innate immunity inflammatory status of many of these diseases. Thus, it has been recognized that there is urgent need for imaging agents that might identify and spatially-temporally localize changes in the activation state of the innate immune system in vivo (Zinnhardt et al., 2018, Jiemy et al., 2018, and Signore et al., 2017).

One essential biochemical marker of the innate immune system is the active production of reactive oxygen (ROS) and nitrogen species (RNS), including superoxide from NADPH oxidase 2 (Nox2), and hypochlorous acid from myeloperoxidases (MPO). MPO, which constitutes 5% of neutrophil dry weight, is concentrated in primary granules (Schultz et al., 1962, Khan et al., 2018), and reactive nitrogen species from inducible nitric oxide synthase (iNOS), are also actively studied biomarkers in a variety of diseases. For example, upon neutrophil activation, primary granules fuse to the phagosomal or cell membrane to produce superoxide from Nox2. Superoxide is then converted to hydrogen peroxide that serves as a substrate for MPO to produce hypochlorous acid (HOCl) (Klebanoff et al., 2013). The Fenton reaction, a catalytic process in the presence of ferrous iron that converts hydrogen peroxide into hydroxyl radicals, may also occur within inflamed tissues (Gross et al., 2009). Of note, in the context of reaction with parasites, Nox2 migrates to the surface of the cells and releases superoxide into the extracellular environment. Reactive oxygen species (ROS) and nitrogen species (RNS) can oxidize apolipoproteins, disrupt endothelial function, and accumulate in the local microenvironment of tumors or the shoulder regions of plaques, suggesting a possible role in atherogenesis (Soehnlein, 2012; Nicholls and Hazen, 2005; Violi et al 2017). Previous studies reviewed elsewhere have shown that circulating MPO levels in the blood correlate with measures of cardiovascular disease severity and predict short- and long-term patient outcomes (Schindhelm et al., 2009). However, the relationship to cancer outcomes is unknown. For these analyses, plasma MPO concentration is usually measured by enzyme-linked immunosorbent assay (ELISA; Chen et al., 2011), which is costly, time-consuming, and typically uses ROS generated by immunoconjugate horseradish peroxidase (HRP) instead of directly measuring MPO-derived ROS. A novel bioluminescence assay, designated MPO activity on a polymer surface (MAPS), for measuring MPO activity in human plasma samples using the bioluminescent substrate L-012 was recently discovered, providing an inexpensive and rapid assay for determining MPO activity in plasma samples from patients with cardiovascular disease or potentially other immune and inflammatory disorders (Goiffon et al., 2015). The method represents an ex vivo assay, lacking in vivo spatial localizing properties required for tumor analysis. L-012 has recently been demonstrated to report on reactive oxygen and nitrogen species in shallow tissues in models of acute contact dermatitis, arthritis, and toxic shock syndrome (Kielland et al., 2009)

Regarding imaging approaches, the present inventors have previously shown that MPO activity can be imaged directly in vivo with luminol, a chemiluminescent compound oxidized by HOCl (Gross et al., 2009). L-012 is a luminol analogue that has also been used to measure ROS and reactive nitrogen species (RNS) in vivo and in vitro with enhanced luminescence and sensitivity (Scheme 1; Daiber et al., 2004 and Kielland et al., 2009). ROS concentrations at inflammation loci are high enough to oxidize bioluminescent probes for real-time whole-animal imaging with charge-coupled device cameras. However, this optical method cannot be applied to humans as the photons released by deep bioluminescent reactions can only travel approximately 1 cm in biological tissues, and thus, not scalable to use in humans. There are pre-clinical magnetic resonance imaging (MRI) reporters for measuring myeloperoxidase activity that involve the activation-mediated polymerization and trapping of complexes containing heavy metals, such as the gadolinium, at the target site to generate MR imaging signals and contrast (Chen et al., 2004, Breckwoldt et al., 2008, and Rodriguez et al., 2010). However, creating an uncontrolled polymerization reaction inside patients as a “non-invasive” readout of peroxidase activity has been questioned, and there are emerging concerns around the chronic toxicity of gadolinium in monomeric chelators, especially at the contrast agent masses required to generate signal by MRI. Radioactive derivatives of dihydroethidium have been synthesized and tested in neuroinflammation models (Hou et al., 2018), but have significant liver retention (Abe et al., 2014), cardiac retention (Zhang et al., 2016), and possibly tumor retention (Owusu-Ansah et al., 2008) as the compound is trapped in non-disease states due to mitochondrial oxidation ultimately leading to lower contrast ratios.

In some aspects, the present disclosure provides novel compounds and kits for creating F-18 imaging agents. Novel fluorine-18 compounds are used to image the activation of the innate immune system. Methods of imaging comprise administering a compound of the present disclosure to a patient. In some embodiments, the method further comprises measuring or detecting a level of an enzyme activity, such as enzymes associated with inflammation. In some embodiments, the enzyme is a peroxidase, such as MPO, an oxidase, such as Nox2 or iNos, or a dual oxidase peroxidase, such as DUOX. In some embodiments, the level of the enzyme may be used to diagnose, prognose, stage, or monitor the progression of a disease or disorder, including but not limited to diseases associated with inflammation. In some embodiments, the activity is higher in diseased cells compared to non-diseased cells. In some embodiments, elevated levels of ROS activity may indicate the presence of a tumor, such as a breast cancer tumor.

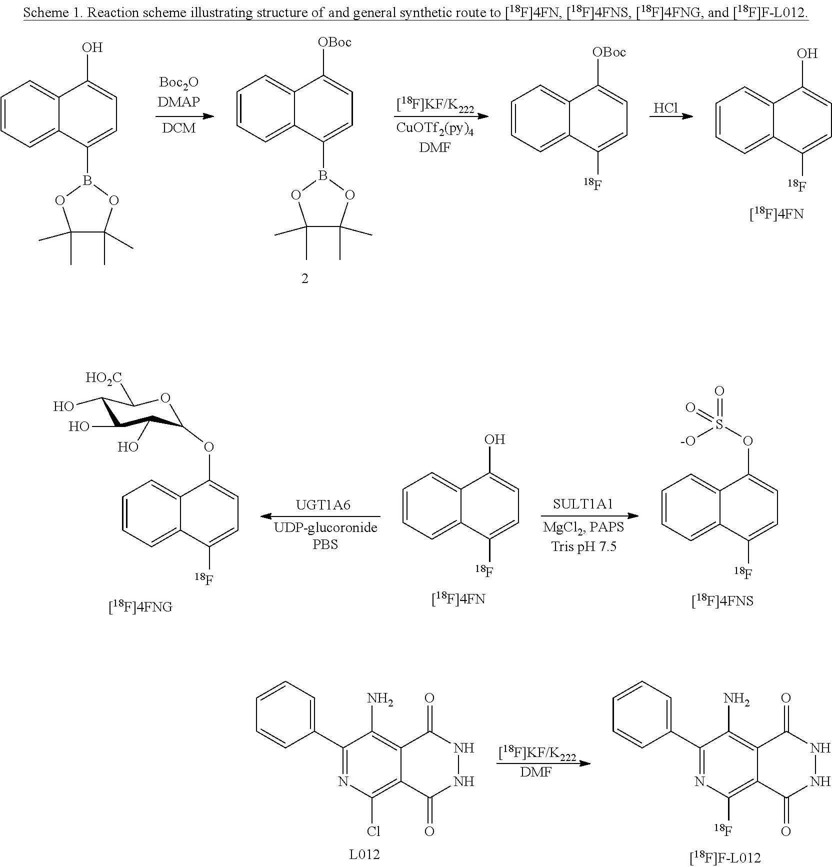

In some aspects, the present disclosure provides noninvasive methods to assay innate inflammation via ROS activity alone or with acid hydrolases in vivo in real time in deep tissues by PET imaging. The radiolabeled compounds of the present disclosure are facile to synthesize as PET radiopharmaceuticals, which can be injected at true tracer levels for imaging, thus minimizing pharmacologic toxicity issues. Furthermore, these radiotracers have been tested in the context of animal models of acute inflammation and tumors for demonstration of efficacy and at safe diagnostic radiation doses that may be scalable to humans. There are many important clinical scenarios that could benefit from rapid, quantitative, non-invasive and repetitive measurements of reactive oxygen and nitrogen species to assess inflammation and immune response in vivo.

### IV. FORMULATIONS AND ROUTES OF ADMINISTRATION

In another aspect, for administration to a patient in need of diagnostic evaluation and/or treatment, radiopharmaceutical formulations (also referred to as radiopharmaceutical preparations, radiopharmaceutical compositions, radiopharmaceuticals, or radiopharmaceutical products) comprise a diagnostically or therapeutically effective amount of a radio-labeled compound disclosed herein formulated with one or more excipients and/or drug carriers appropriate to the indicated route of administration.

In some embodiments, the radiolabeled compounds disclosed herein are formulated in a manner amenable for diagnostic evaluation or treatment of human and/or veterinary subjects.

In some embodiments, formulation comprises admixing or combining one or more of the radiolabeled compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Radiopharmaceutical formulations may be administered by a variety of methods, such as by injection (e.g., subcutaneous, intravenous, and intraperitoneal). Depending on the route of administration, the radiolabeled compounds disclosed herein may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes. The radiolabeled compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Radiopharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. Radiotracers, particularly ROS sensors, may be subject to radiolysis and proper stability can be maintained with antioxidants stabilizers, for example, by the use of sodium ascorbate in the formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of radiopharmaceutical calculated to produce the desired imaging effect in association with the required pharmaceutical carrier. In some embodiments, the specification for the dosage unit forms of the disclosure are dictated by and directly dependent on (a) the unique characteristics of the radiopharmaceutical and the particular imaging effect to be achieved, and (b) the limitations inherent in the art of compounding such a radiopharmaceutical for the imaging of a patient. In some embodiments, active compounds are administered at an effective dosage sufficient to produce a PET image of immune activity in a patient. For example, the efficacy of a compound can be evaluated in an animal model system that may be predictive of efficacy in imaging immune activity in a human or another animal.

In some embodiments, an imaging agent comprising an isotope such as a radioisotope may be referred to as being “isotopically enriched.” An “isotopically enriched” composition refers to a composition comprising a percentage of one or more isotopes of an element that is more than the percentage (of such isotope) that occurs naturally. As an example, a composition that is isotopically enriched with a fluoride species may be “isotopically enriched” with fluorine-18 (¹⁸F). Thus, with regard to a plurality of compounds, when a particular atomic position is designated as ¹⁸F, it is to be understood that the abundance (or frequency) of ¹⁸F at that position (in the plurality) is greater, including substantially greater, than the natural abundance (or frequency) of ¹⁸F, which is essentially zero. In some embodiments, a fluorine designated as ¹⁸F may have a minimum isotopic enrichment factor of about 0.001% (i.e., about 1 out of 10⁵ fluorine species is ¹⁸F), 0.002%, 0.003%, 0.004%, 0.005%, 0.006%, 0.007%, 0.008%, 0.009%, 0.01%, about 0.05%, about 0.1%, about 0.2%, about 0.3%, about 0.4%, about 0.5%, about 0.75%, about 1%, about 2%, about 3%, about 4%, about 5%, about 10%, about 15%, about 20%, about 30%, about 40%, about 50%, about 60%, about 70%, about 80%, about 90%, about 95%, or greater. The minimum isotopic enrichment factor, in some instances, may range from about 0.001% to about 1%. The isotopic enrichment of the compounds provided herein can be determined using conventional analytical methods known to one of ordinary skill in the art, including mass spectrometry and HPLC.

In some embodiments, the effective radioactivity dose range for the radiolabeled compound can be extrapolated from effective doses determined in animal studies for a variety of different animals. In some embodiments, the radiolabeled compound is administered by intravenous injection, usually in saline solution, at a dose of between about 0.1 and about 50 mCi (and all combinations and subcombinations of dosage ranges and specific dosages therein), and as described below. Precise amounts of the radiolabeled compound depend on the judgment of the practitioner and are specific to each individual. Imaging is performed using techniques well known to the ordinarily skilled artisan and/or as described herein.

The maximum desirable dose administered to a subject may be based on determining the amount of radiolabeled compound of the present disclosure (e.g., [¹⁸F]4FN), which limits the radiation dose to about 5 rem to the critical organ (e.g., urinary bladder) and/or about 1 rem effective dose (ED) or lower, as will be understood by those of ordinary skill in the art. In some embodiments, the maximum desirable dose or total amount of radiolabeled compound administered is between about 1 mCi and about 20 mCi. In some embodiments of the disclosure, the maximum desirable dose or total amount of radiolabeled compound administered is between about 5 mCi and about 15 mCi. In some embodiments of the disclosure, the maximum desirable dose or total amount of radiolabeled compound administered is between about 8 mCi and about 12 mCi. In some embodiments, a desirable dose may be less than or equal to about 15 mCi, less than or equal to about 14 mCi, less than or equal to about 13 mCi, less than or equal to about 12 mCi, less than or equal to about 11 mCi, or less than or equal to about 10 mCi over a period of time of up to about 10 minutes, about 30 minutes, about 1 hour, about 2 hours, about 6 hours, about 12 hours, about 24 hours, or about 48 hours.

In some embodiments, the total amount of radiolabeled compound administered to a subject is between about 0.1 mCi and about 30 mCi, or between about 0.5 mCi and about 20 mCi. In some embodiments, the total amount of radiolabeled compound administered to a subject is less than or equal to about 50 mCi, less than or equal to about 40 mCi, less than or equal to about 30 mCi, less than or equal to about 20 mCi, less than or equal to about 18 mCi, less than or equal to about 16 mCi, less than or equal to about 15 mCi, less than or equal to about 14 mCi, less than or equal to about 13 mCi, less than or equal to about 12 mCi, less than or equal to about 10 mCi, less than or equal to about 8 mCi, less than or equal to about 6 mCi, less than or equal to about 4 mCi, less than or equal to about 2 mCi, less than or equal to about 1 mCi, or less than or equal to about 0.5 mCi. The total amount administered may be determine based on a single dose or multiple doses administered to a subject within a time period of up to or at least about 30 seconds, about 1 minute, about 10 minutes, about 30 minutes, about 1 hour, about 2 hours, about 6 hours, about 12 hours, about 24 hours, about 48 hours, or about 1 week.

In some embodiments, between about 0.1 and about 30 mCi of radiolabeled compound is administered to a subject, and a first period of image acquisition begins at the time of administration (e.g., injection) or begins at more than about 0 minutes, about 1 minute, about 2 minutes, about 3 minutes, about 4 minutes, about 5 minutes, prior to the administration of the radiolabeled compound. In some embodiments, the first imaging continues for at least about 5 minutes, about 10 minutes, about 15 minutes, about 30 minutes, about 45 minutes, about 60 minutes, about 75 minutes, about 90 minutes, about 105 minutes, about 120 minutes, or longer. Following the first period of imaging, the subject may undergo one or more additional imaging acquisition periods during up to about 1, about 2, about 3, about 4, about 5, about 6, or more hours after the administration of radiolabeled compound. One or more additional image acquisition periods may have a duration of between about 3 and about 40 minutes, about 5 and about 30 minutes, about 7 and about 20 minutes, about 9 and about 15 minutes, and may be for about 10 minutes. The subject, in some embodiments, may return once, twice, or three or more times for additional imaging following the first injection of radiopharmaceutical compound wherein a second, third, or more, injections of radiopharmaceutical compound may be administered. A non-limiting example of an administration and image acquisition method for radiopharmaceutical compound for a subject comprises injection of between about 0.1 and about 30 mCi of radiopharmaceutical compound to the subject, with image acquisition starting less than about 10 minutes before the injection and continuing for about 60 minutes. In some embodiments, the subject undergoes first or additional image acquisition for about 10 minutes, or for about 20 minutes, or for about 30 minutes, or for about 40 minutes, or for about 50 minutes, or for about 60 minutes, at about one hour, or about two hours, or about 3 hours, or about 4 hours, and at about 4 hours, or about 5 hours, or about 6 hours, or about 7 hours, or about 8 hours, after the injection of radiopharmaceutical compound.

Principles and techniques for radiopharmaceutical dosimetry are taught, for example, in Zanzonico, 2000, which is incorporated by reference herein. Other factors affecting the dose include the physical and clinical state of the patient, the route of administration, the intended goal of imaging, and the stability and toxicity of the particular radiopharmaceutical formulation. The actual dosage amount of a compound of the present disclosure or composition comprising a compound of the present disclosure administered to a patient may be determined by physical and physiological factors such as type of subject treated, age, sex, body weight, severity of condition, the type of disease being imaged, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

### V. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thio” means=S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “-̅-̅-̅-̅” represents a single bond or a double bond. Thus, the formula

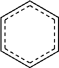

covers, for example

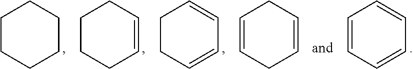

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

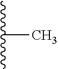

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

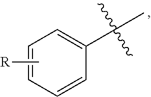

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

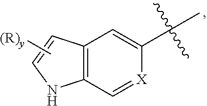

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

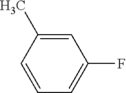

is also taken to refer to

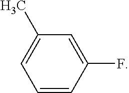

Aromatic compounds may also be depicted using a circle to represent the delocalized nature of the electrons in the fully conjugated cyclic π system, two non-limiting examples of which are shown below:

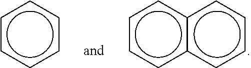

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

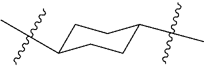 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

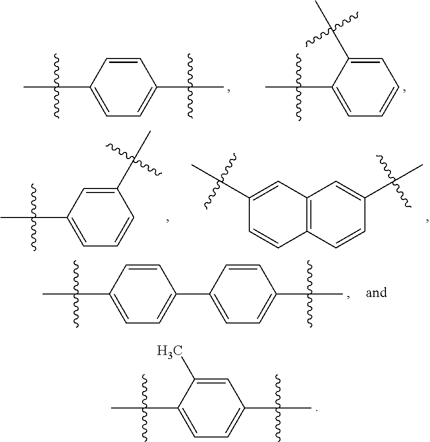

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

The terms “alkylsulfonyl” and “alkylsulfinyl” refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “alkoxysulfonyl”, “alkylaminosulfonyl”, “dialkylaminosulfonyl”, “heteroarylsulfonyl”, and “heterocycloalkylsulfonyl” are defined in an analogous manner.

An “amine protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monovalently protected amino group” and PG_{DA} is a divalent amine protecting group as described above, which may also be described as a “divalently protected amino group”.

A “boronic acid protecting group” is well understood in the art. A boronic acid protecting group is a group which prevents the reactivity of the boronic acid group during a reaction which modifies some other portion of the molecule and can be easily removed to generate the desired boronic acid. Boronic acid protecting groups can be found at least in Greene and Wuts, 1999, which is incorporated herein by reference. Non-limiting examples of boronic acid protecting groups include those derived from the reaction of a boronic acid with 1,2-diols, such as pinacol or pinanediol, and those derived from the reaction of a boronic acid with a diacid, such as N-methyliminodiacetic acid. When used herein, a protected boronic acid group is a group of the formula —OPG_{B}O— wherein PG_{B} is a boronic acid protecting group as described above.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfate groups such as —S(O)₂OH and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the formula PG_{H}O— wherein PG_{H} is a hydroxyl protecting group as described above.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a radiopharmaceutical) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” in the context of imaging a patient means that amount of the compound or composition which, when administered to the subject or patient, is sufficient to produce an image, such as a PET image. “Effective amount,” “therapeutically effective amount” or “pharmaceutically effective amount” when used in the context of treatment, therapy, prevention or diagnosis, means that amount of the compound which, when administered to a subject or patient, is sufficient to effect such treatment, therapy, prevention, or diagnosis, respectively.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrofluoric acid, hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient or radiolabeled compound that is involved in carrying, delivering and/or transporting the ingredient or compound. Drug carriers may be used to improve the delivery and the effectiveness of drugs or diagnostic agents, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active agent according to the present disclosure. The prodrug itself may or may not also have activity with respect to a given target protein. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-O-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

As used herein the term “radiolabeled-compound” does not include any single molecule of the formula or any group of molecules of the formula wherein the indicated radioisotope of the formula is present in its natural isotopic abundance.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Imaging” includes any technique and processing method of creating visual representations of the interior of a body for clinical analysis and medical intervention, as well as visual representation of the physiological or biochemical function of some organs or tissues.

“Positron emission tomography (PET) scanning” is a nuclear imaging technology (also referred to as molecular imaging) that enables visualization of the fate of a radiopharmaceutical in deep tissues in real time in living subjects. PET detects pairs of annihilation photons emitted by a positron-emitting radionuclide incorporated into a small molecule, peptide, protein or nanoparticle that contains moieties that confer targeting capacity to the entity and visualized from inside a living subject. The radionuclide and targeted carrier together are called a radiopharmaceutical or radiotracer.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single diagnostically effective dose of the active ingredient to a patient in a single administration. Such unit dose formulations that may be used include but are not limited to a single tablet, capsule, or other oral formulations, or a single vial with a syringeable liquid or other injectable formulations.

The term “kit” refers to forms of matter comprising a ready-to-use solution, powder, or gel, for the production of a radiopharmaceutical of the present disclosure, one may use the aqueous infusible and injectable solutions known for this purpose, optionally together with the excipients, carriers and/or stabilizing substances known in the art.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Radiotracers and Use in Imaging

A. Design, Synthesis, and Characterization of Radiotracers

Synthesis of [¹⁸F]4-Fluoronaphthol ([¹⁸F]4FN)

### Synthesis of the Precursor 2:

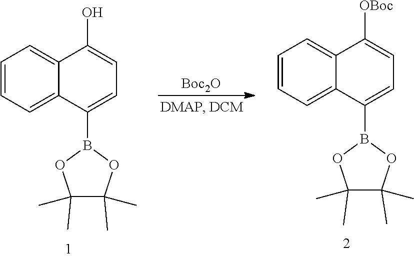

Commercially available 4-(4,4,5,5-tetramethyl-1,3,2-dioxaborolan-2-yl)naphthalen-1-ol (ChemBridge, San Diego, Calif.) (1, 100 mg, 0.37 mmol) was dissolved in dichloromethane (1.5 mL) and Boc₂O (81 mg, 0.37 mmol) and DMAP (45 mg, 0.37 mmol) were added in turn. The mixture was stirred at rt overnight, solvent removed, and the crude residue purified on silica gel (Biotage, % AcOEt in Hexanes: 0% for 4 CV, 0%→20% in 5 cv, 20% for 2 cv). Naphthol 2 (110 mg) was obtained as a white solid in 80% yield.

2: ¹H NMR (500 MHz, Chloroform-d) δ 8.84-8.76 (m, 1H), 8.08 (d, J=7.6 Hz, 1H), 8.00 (dt, J=8.1, 1.0 Hz, 1H), 7.58-7.49 (m, 2H), 7.32 (d, J=7.6 Hz, 1H), 1.58 (s, 9H), 1.41 (s, 12H). ¹³C NMR (126 MHz, CDCl₃) δ 151.6 (s), 149.6 (s), 138.4 (s), 135.6 (d), 128.6 (d), 126.9 (d), 126.6 (d), 126.0 (s), 121.2 (d), 116.9 (d), 83.8 (s), 83.7 (s, 2C), 81.0 (s), 27.8 (q, 3C), 24.9 (q, 4C). MS: m/z (%)=393 (40) [MNa]⁺, 271 (75).

### Synthesis of Boc-4-Fluoro-1-Naphthol 3:

Boc-4-fluoro naphthol 3 has been synthesized by following the same procedure reported for precursor 2.

3: ¹H NMR (500 MHz, Chloroform-d) δ 8.20-8.12 (m, 1H), 8.05 (dd, J=7.0, 2.5 Hz, 1H), 7.60-7.52 (m, 2H), 6.98 (dd, J=10.2, 8.1 Hz, 1H), 6.71 (dd, J=8.2, 4.0 Hz, 1H), 5.23 (s, 1H).

Synthesis of [¹⁸F]4FN ([¹⁸F]4)

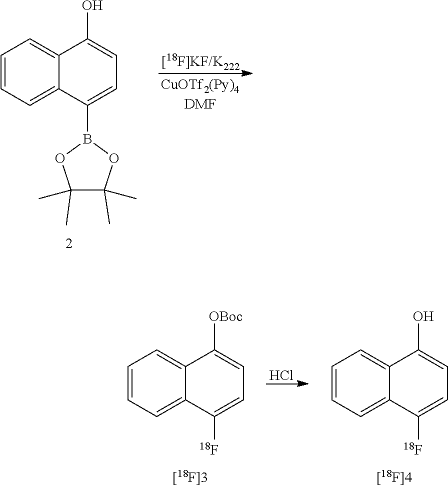

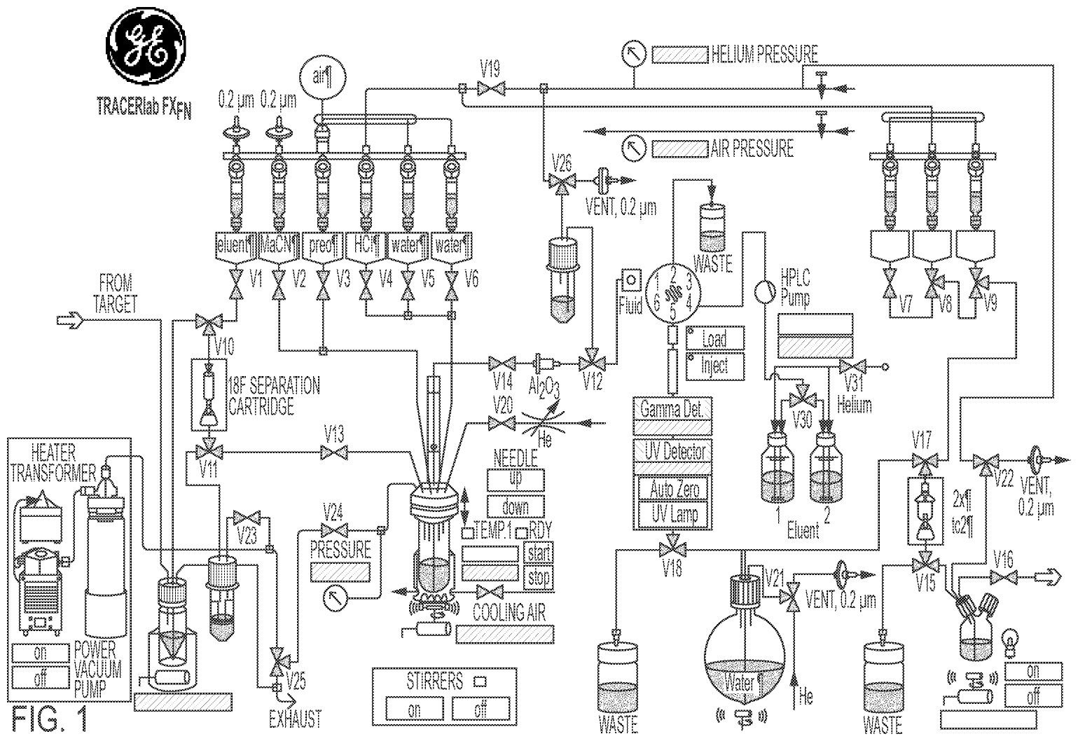

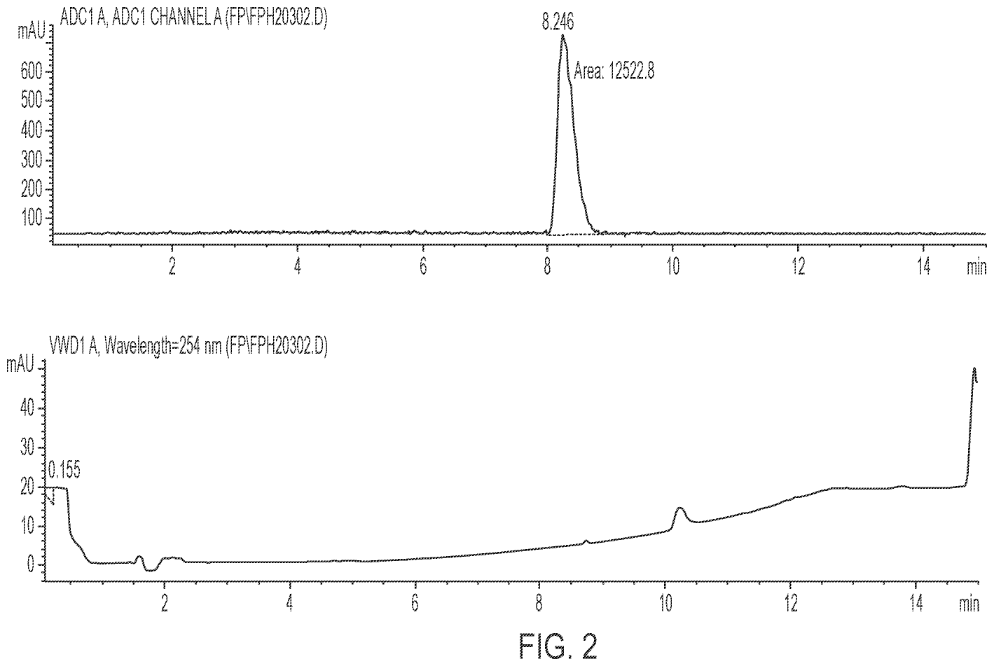

The radiosynthesis was performed on a TracerLab FX (General Electric Healthcare, Munster, Germany) automatic module. [¹⁸F]Fluoride was obtained as an aqueous solution from the MD Anderson Cyclotron Radiochemical Facility (CRF). [¹⁸F]Fluoride was adsorbed on an ion exchange cartridge (Pre-conditioned Sep-PAK® Light QMA Cartridge, ABX GmbH, Radeberg, Germany). [¹⁸F]Fluoride was flushed into the reaction vial with a potassium carbonate and Kryptofix 2.2.2. water/CH₃CN solution (700 μL; stock solution: 52.8 mg of K₂CO₃, 240.1 mg of K₂₂₂, 4 mL of water, 16 mL of CH₃CN). The solution was dried under vacuum and under nitrogen flow at 60° C. for 2 min. 500 μL of dry CH₃CN was added and then the mixture was azeotropically dried at 120° C. for additional 3 min. Synthesis of 4-[¹⁸F]fluoro-1-naphthol was carried out by adding Boc naphthol boronate 2 (6-11 mg) and CuOTf₂(Py)₄ (12-23 mg) in dry DMF (600 μL) to the dried [¹⁸F]fluoride. Air was allowed to enter into the reactor by equipping vial 3 with a balloon filled with compressed air (see Tracerlab, FIG. 1 below). The mixture was stirred at 110° C. for 20 min, then cooled down at 80° C. and HCl 1M (1 mL) was added. The mixture was stirred at 80° C. for 10 min, cooled down at 30° C. and diluted with water (3 mL). The crude was purified by semi preparative HPLC (Luna 5 μm C18(2) 100 Å, 250×10 mm) eluting with a 50% MeCN/water (0.085% H₃PO₄) [rt=15 min]. The radioactive product was collected into the Tracerlab collection flask pre-filled with water (50 mL). The solution was loaded onto two-in series tC2 cartridges (Sep-PAK®, Waters, Milford, USA). The cartridges were washed with 6 mL of water, dried under nitrogen and eluted with ethanol. The overall synthesis time was approximately 70 min. Activity was determined by dose calibrator and a sample was taken for quality control (QC). QC was performed by analytical radio-HPLC (Agilent 1100 equipped with a single wavelength UV detector and an in-line Bioscan Inc. B-FC-4100 gamma detector) on a C8 column (Agilent ZORBAX, Eclipse XDB-C8, 4.6×150 mm, 5 μm, a water (0.1% (v/v) TFA) and CH₃CN (0.1% (v/v) TFA) gradient (40% B for 3 min, 40% B→95% B in 7 min, 95% B for 4 min) with a flow of 1 mL/min. The identity of the radiolabeled compound was confirmed by co-elution of the original cold standard (Toronto Research Chemicals, Toronto, Ontario, Canada). The HPLC trace of purified [¹⁸F]4FN is shown in FIG. 2.

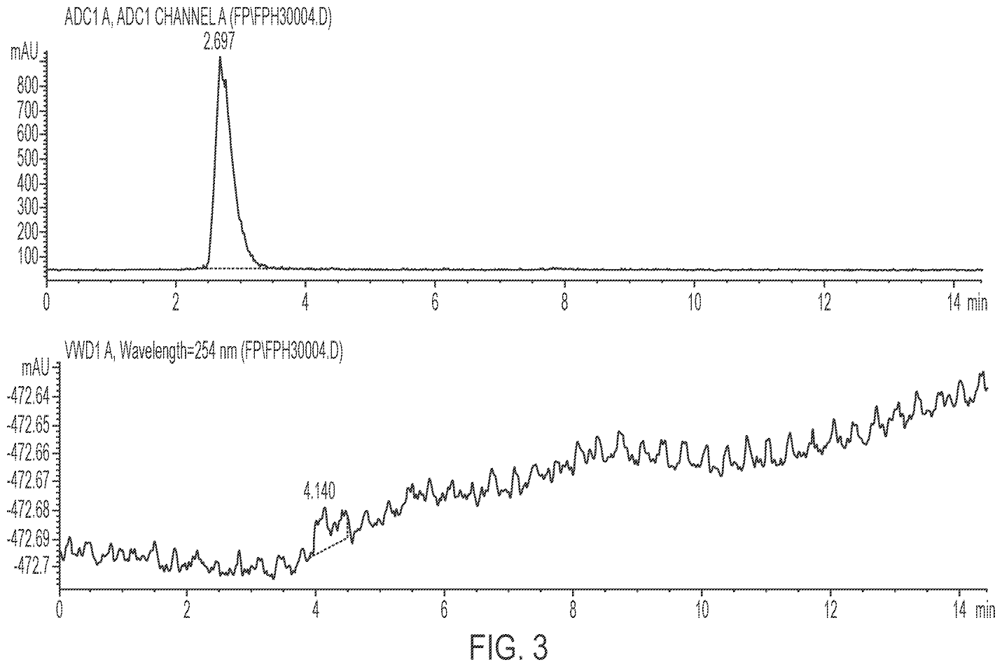

### Synthesis of [¹⁸F]4FNS

[¹⁸F]FN in ethanol (100 μL) was added to a solution containing 50 mM Tris pH-7.5, 15 mM MgCl₂, PAPS 100 μM, and recombinant human SULT1A1 (5 μg/mL) (2 mL) and incubated for 1 hour at 37° C. Ethanol was maintained at <5%. An aliquot was removed and QC was performed on radio-HPLC with inline gamma detector and UV detector on a C18 column. Reaction was >99% complete from [¹⁸F]FN with no evidence of fluoride contamination. The solution was diluted with MeCN/H₂O (2 mL), passed through a silica cartridge (Alltech, Houston, USA) and purified by semi preparative HPLC (Luna 5 μm C18(2) 100 Å, 250×10 mm) eluting with a 50% MeCN/water (0.085% H₃PO₄) [rt=10 min]. The radioactive product was collected into the Tracerlab collection flask pre-filled with water (10 mL). The solution was loaded onto a light C18 cartridge (Sep-PAK®, Waters, Milford, USA). The cartridge was washed with 6 mL of water, dried under nitrogen and eluted with 1 mL of ethanol. The overall synthesis time was approximately 80 min. Activity was determined by dose calibrator and a sample was taken for quality control (QC). QC was performed by analytical radio-HPLC (Agilent 1100 equipped with a single wavelength UV detector and an in-line Bioscan Inc. B-FC-4100 gamma detector) on a C8 column (Agilent ZORBAX, Eclipse XDB-C8, 4.6×150 mm, 5 μm, a water (0.1% (v/v) TFA) and CH₃CN (0.1% (v/v) TFA) gradient (40% B for 3 min, 40% B→95% B in 7 min, 95% B for 4 min) with a flow of 1 mL/min. [¹⁸F]4FNS was obtained in (non-optimized) 0.62±0.12 decay corrected radiochemical yield (n=3). The HPLC trace of purified [¹⁸F]4FNS is shown in FIG. 3.

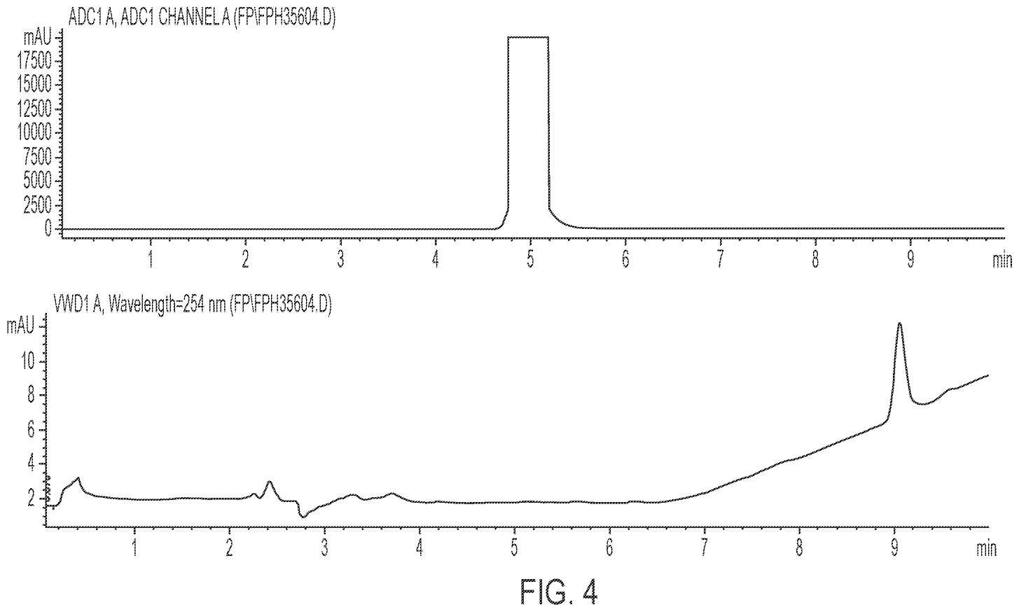

### Synthesis of [¹⁸F]4FNG

[¹⁸F]FN in ethanol (10 μL) was added to a solution phosphate buffered saline+0.5 mg/mL human UGT1A6 “Supersome” Corning+2 mM UDP-glucoronide. (200 μL) and incubated for 1 hour at 37° C. Ethanol was maintained at <5%. An aliquot was removed and QC was performed on radio-HPLC with inline gamma detector and UV detector on a C18 column. Reaction was >99% complete from [¹⁸F]4FN with no evidence of fluoride contamination. [¹⁸F]FNG was purified by semi preparative HPLC (Luna 5 μm C18(2) 100 Å, 250×10 mm) eluting with a 50% MeCN/water (0.085% H₃PO₄) [rt=6 min]. QC was performed by analytical radio-HPLC (Agilent 1100 equipped with a single wavelength UV detector and an in-line Bioscan Inc. B-FC-4100 gamma detector) on a C18 column (Econosil C18 10 μm 250 mm×4.6 mm), a water (0.1% (v/v) TFA) and CH₃CN (0.1% (v/v) TFA) gradient (40% B for 3 min, 40% B→95% B in 7 min, 95% B for 4 min) with a flow of 1 mL/min. The HPLC trace of purified [¹⁸F]4FNG is shown in FIG. 4.

B. In Vivo Study Design and Methodology

### 4T1 Tumor Model Implantation

4T1 murine breast cancer cells were cultured and passaged per ATCC guidelines and utilized prior to 20 passages. 10,000 4T1 cells were injected subcutaneously into the right flank of 6-8 week old Balb/c mice. Tumors grew for 3 weeks prior to imaging.

### Ear-Inflammation Animal Model Imaging and Analysis

3 Months old Balb/c female mice or 20-26 week-old female C57B16 mice (Taconic) were topically treated with PMA (Sigma) (400 μM) in ethanol on the right ear or vehicle, ethanol, on the left ear. 24 hrs later, inflammation of the right ear was validated by visual inspection for redness and swelling. Mice were then injected IV (unless noted) with [¹⁸F]FN or [¹⁸F]FNS or [¹⁸F]FNG derivatives as noted and PET/CT images were acquired at times indicated, typically 1 hr post injection. Mice, n=3 for [¹⁸F]FN and [¹⁸F]FNS, were euthanized at 1.5 hr post injection for blood metabolism analysis. In one experiment, adult (3 month old Balb/c) were imaged on an in vivo imaging system (IVIS Spectrum, PE; 5 min, binning 16, 25 cm FOV) 10 min post injection of Luminol (200 mg/kg) IP to confirm production of reactive oxygen species. For [¹⁸F]FDG PET imaging experiments, mice were fasted for >4 hr prior to FDG injection. Mice were injected with [¹⁸F]FDG IV under light anesthesia and then allowed to awake, move and drink water at libitum. Cages were kept on a cage warmer during uptake and washout periods. Mice were imaged starting at 45 min post-injection with a 10 min PET/CT scan (Albira, Bruker).

In all cases, PET images were acquired for 10 min using a 15 cm FOV; CT images were acquired for fusion using a 7 cm FOV also centered on the object of interest. Image data were decay corrected to injection time (Albira, Bruker) and expressed as % ID/cc or SUV as indicated (PMOD, PMOD Technologies). Actual injected dose was calculated based on measuring the pre- and post-injection activity in the syringe with a dose calibrator on a per mouse basis (Capintec), and mice were individually weighed to calculate individual standardized uptake values.

C. Results and Discussion

[¹⁸F]4FN was synthesized on the automated module GE TracerLab-FX from precursor 1 in 6.8±2.5% (n=22) activity yield, by means of a copper-mediated radiofluorination of the corresponding Boc-protected pinacol boronic ester, followed by deprotection, purification and reformulation (Scheme 2; Tredwell et al., 2014). [¹⁸F]4FN was obtained in >99% radiochemical purity, 50-140 GBq/μmol and up to 4 h shelf-stability in PBS (10% EtOH). [¹⁸F]4FN was also stable in mouse plasma for at least 1 h.

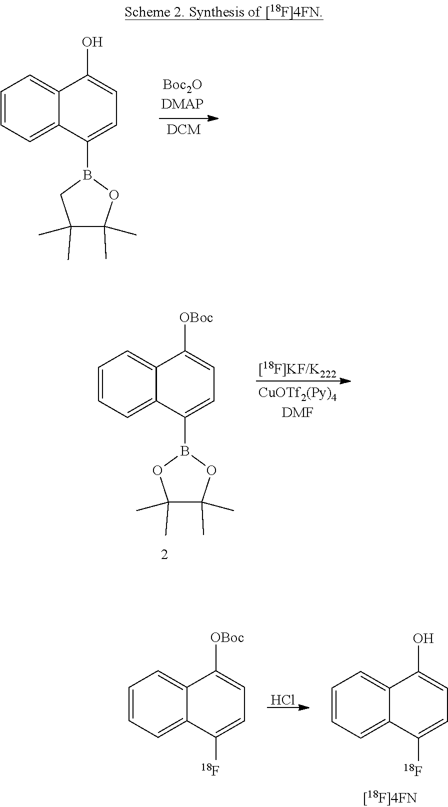

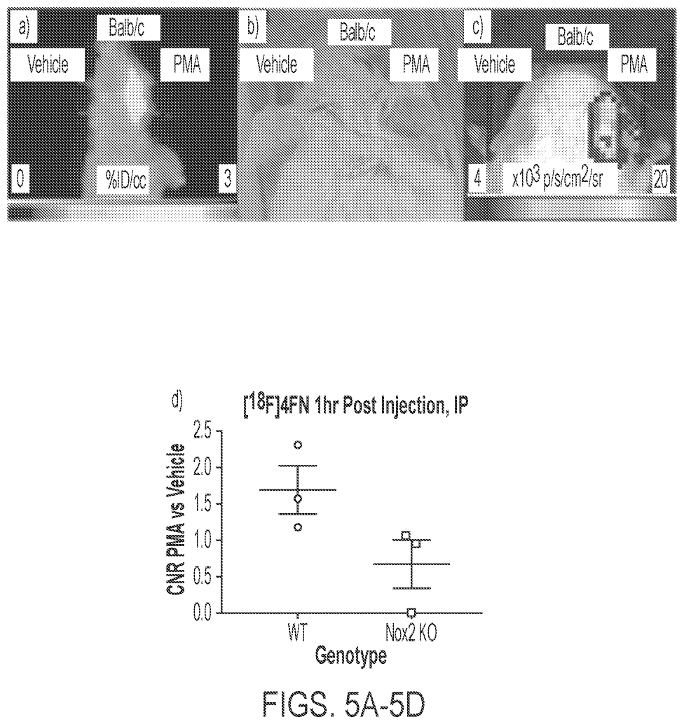

In the context of an acute inflammation model, [¹⁸F]4FN was tested in a pilot experiment in Balb/c mice topically treated with PMA on the right ear or vehicle on the left ear. The treated ear turned red, visually confirming the inflammation site. Mice were injected IP with [¹⁸F]4FN and PET/CT scans were taken at 11 min, 1 h, 2 h and 3 h post injection of tracer (FIG. 5). At 11 min post-injection, no differentiation in uptake between the inflamed right ear and the vehicle treated left ear (internal negative control) could be observed. After 1 h, the inflamed ear was clearly visualized (>2% ID/cc). Clearance of the tracer was surprisingly renal dominant as evidenced by the high kidney uptake and bladder excretion. Radioactivity in the liver, albeit low, could be seen, therefore accounting for the observed partial hepatobiliary excretion. Given the low radioactivity observed in the intracranial region, this tracer may not cross an intact blood-brain barrier. The uptake-ratio between the right-inflamed ear and the left-control ear increased over time. Production of reactive oxygen species in the inflamed ear was confirmed by BLI with luminol after PET imaging (FIG. 5) (Gross et al., 2009). This indicated the dependence of the signal on induced inflammation.

To rule out any differential retention due to differences in perfusion/delivery and non-oxidase targeted retention of this small molecule PET tracer, another small molecule that primarily reports on changes in perfusion, the non-oxidase targeted ⁶⁸Ga-citrate, was injected into the same animals and images were taken at the same time points (Nanni et al., 2010). No difference between the ears was observed at any analyzed time point. To test ROS-dependent trapping of [¹⁸F]4FN, age-matched Nox2 KO mice (gp91phox^{-/-}, Jackson Laboratories) were imaged at 1 post injection (FIG. 5D). Although a small differentiation between the inflamed and non-inflamed ear could be measured, the differences between ears were substantially smaller compared to WT inflamed mice and, furthermore, the small difference decreased over time. The difference in uptake between WT and Nox2 KO mice accounted for oxidase/peroxidase enzyme-mediated trapping of the PET agent. Furthermore, indeed, oxidases/peroxidases are not the only enzymes active during innate immunity; other oxidases, such as eosinophil peroxidase (EPX) could partially trap [¹⁸F]4FN through a similar oxidation mechanism, which will be further explored. Moreover, lysosomal hydrolases, including arylsulfatases, glucoronidases, phosphatases, etc., are activated during innate immune response and might contribute to additional signal by further enhancing the inflammation-selective trapping of metabolites (Acharya ett al., 2014, Yin et al., 2018, Hager et al., 2010, Henson, 1971, Szmigielski et al., 1974, and Goetzl, 1976).

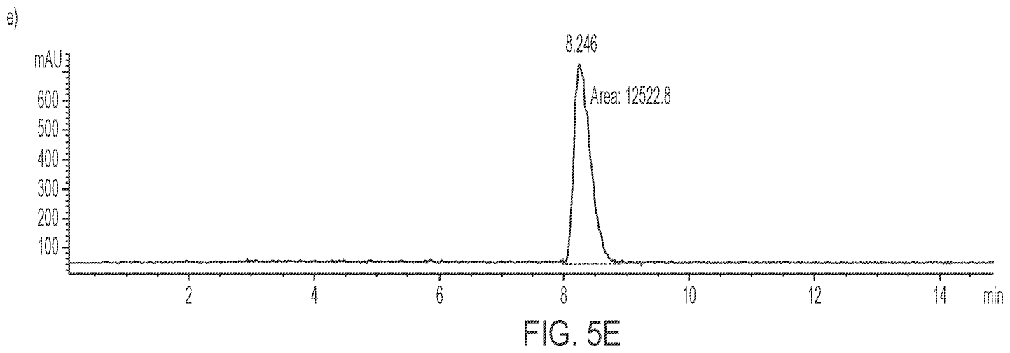

The quantitative selectivity of [¹⁸F]4FN was tested using a PMA ear inflammation model in a larger (n=5) cohort and found that using either IP or IV delivery routes in these mice yielded statistically detectable differences in SUV between inflamed and vehicle-treated ears (FIGS. 6A & 6B). These yielded either strong contrast ratios, which would be easily detectable by eye against non-inflamed tissues, and large Cohen's d effect sizes for testing the difference between inflamed and non-inflamed sites (FIG. 6C).

In vivo mouse blood metabolism was also performed. Blood was drawn from inflamed, non-inflamed and MPO knock-out mice at 5 min and 1.5 h post injection, processed and analyzed by analytical radio-HPLC. At 1.5 h post injection, more polar metabolites were observed in the blood of inflamed mice, whereas in the other cohorts, after 1.5 h, the parent compound could still be seen. Metabolites are also present 5 min after injection, accounting for fast metabolism. While 4-fluoronaphthol metabolism has not been studied, naphthol metabolism is well documented in the literature: the molecule is reported to be quickly transformed into glucuronic and sulfuric esters, and this pathway is conserved from flies to man (Narukawa et al., 2004 and Terriere et al., 1961). It was hypothesized that [¹⁸F]4FN follows the same type of metabolism, which would account for the high renal clearance, because such metabolites would be expected to be more hydrophilic. To test this hypothesis, [¹⁸F]4FN was transformed enzymatically with glucuronosyltransferase and sulfonyl transferase into the corresponding glucuronide and sulfate derivatives (Scheme 3), respectively, and the products analyzed by radio-HPLC. Indeed, the polar metabolites observed in the blood co-elute with the [¹⁸F]4-fluoronaphthalen-1-yl sulfate ([¹⁸F]4FNS) and [¹⁸F]4-fluoronaphthalen-1-yl glucuronide ([¹⁸F]4FNG). Samples of urine were also taken from WT inflamed mice and the HPLC analysis showed the presence of both [¹⁸F]4FNS and [¹⁸F]4FNG.

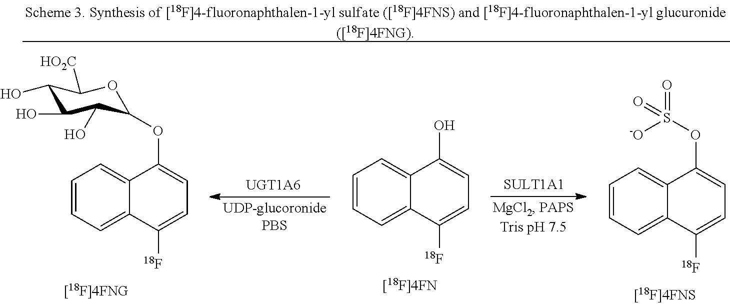

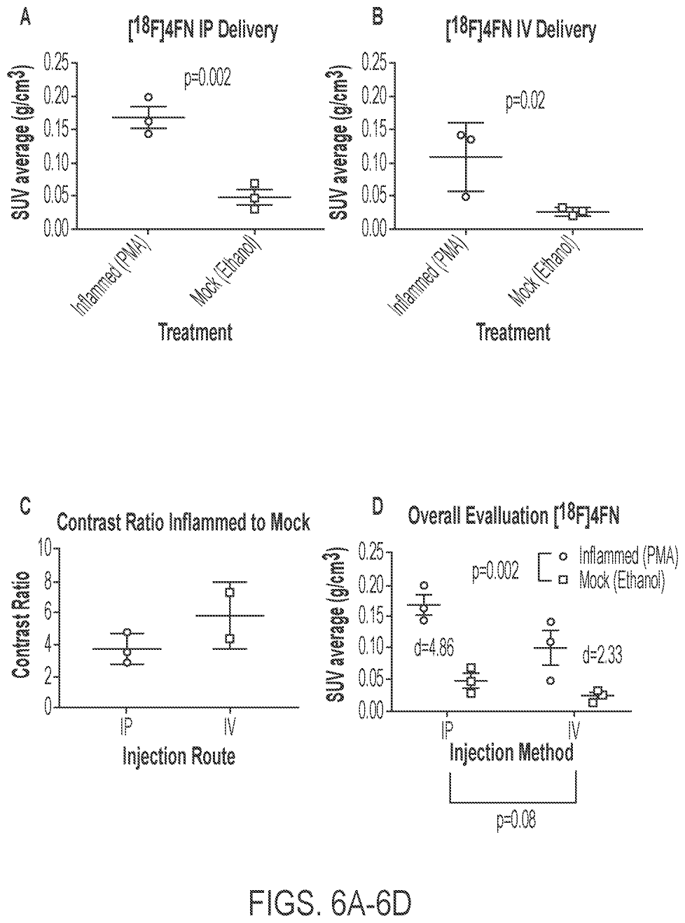

To understand the contribution of [¹⁸F]4FNS to the radioactivity accumulation at the site of inflammation in vivo, [¹⁸F]4FNS was radiosynthesized enzymatically from [¹⁸F]4FN and injected IV using the same PMA ear inflammation model as above (n=3, FIG. 7). The site of inflammation was again readily detected by PET (FIG. 7). Rapid primarily renal clearance was observed with minor hepatobiliary excretion and minor defluorination (bone). Blood analysis showed two peaks corresponding to [¹⁸F]4FNS and [¹⁸F]4FNG, and their ratio was mouse-dependent. The species excreted in urine appeared to be solely [¹⁸F]4FNG, suggesting that [¹⁸F]4FNS can be interconverted into [¹⁸F]4FNG in vivo, the latter being rapidly renal excreted.

Finally, the uptake of [¹⁸F]4FN and [¹⁸F]4FNS was compared with [¹⁸F]FDG, the most extensively used PET imaging tracer in nuclear medicine, in our PMA model of inflammation. [¹⁸F]FDG has been employed to assess inflammation with less-than-ideal results, especially in cancer patients, where it can yield false-positive images (Wu et al., 2013).

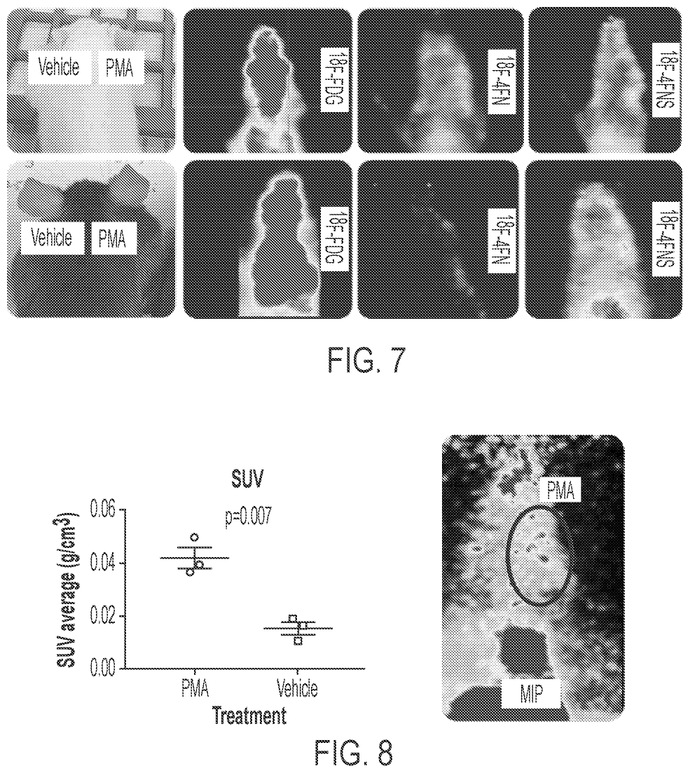

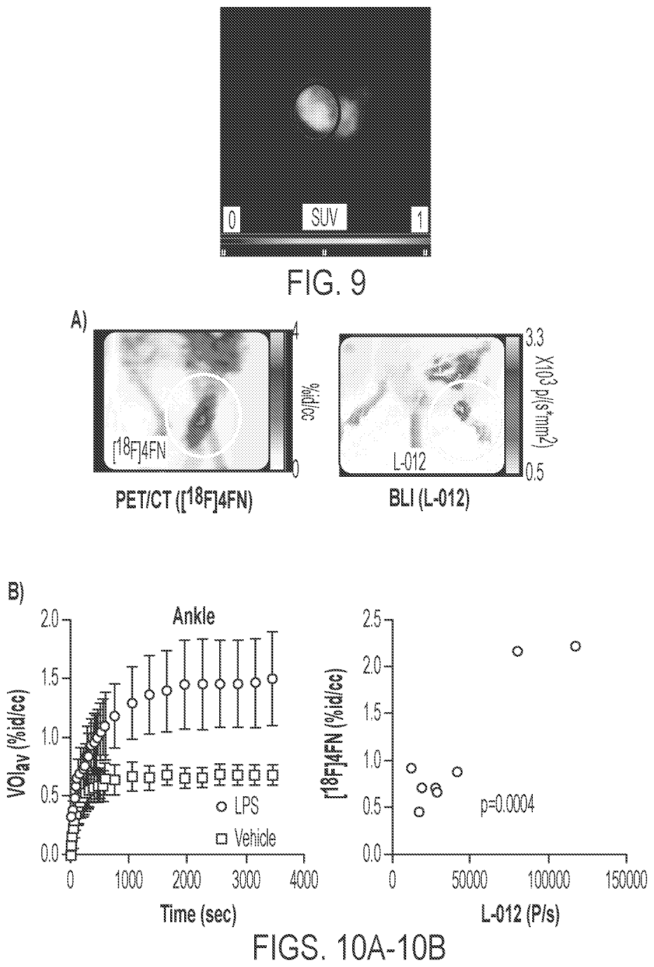

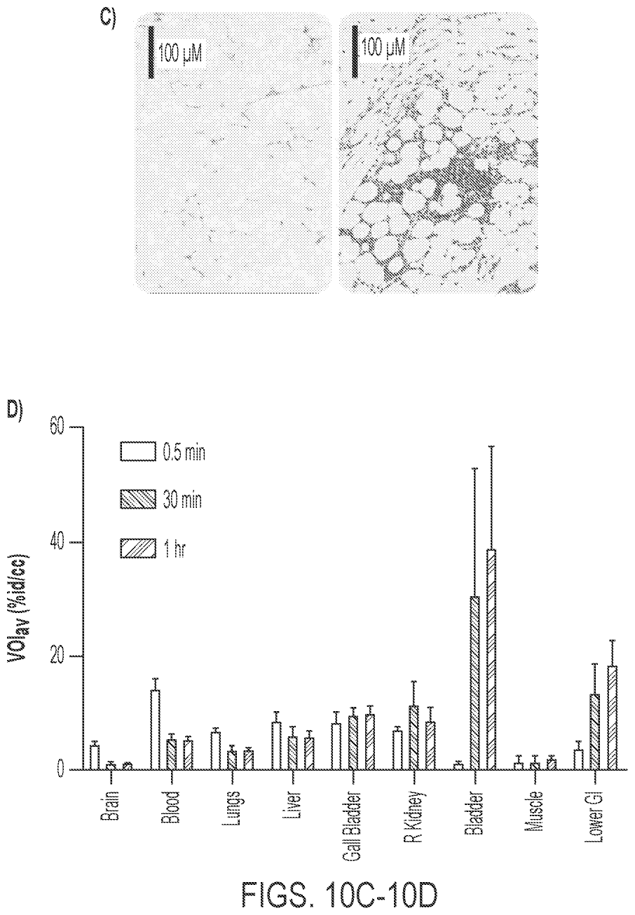

As shown in FIG. 7, as expected, the PMA-inflamed site is clearly visible by [¹⁸F]4FN and [¹⁸F]4FNS PET, but there is no differentiation between the ears with [¹⁸F]FDG PET.

[¹⁸F]FNG also has been directly tested in the same PMA model of inflammation. Although a small differentiation between the PMA-inflamed site and the vehicle-treated ear could be detected, [¹⁸F]FNG was retained much less than [¹⁸F]FN and [¹⁸F]FNS, and also cleared much faster (FIG. 8).

[¹⁸F]4FNS PET has been employed to visualize 4T1 tumors in immunocompetent Balb/C mice. 4T1 is a reliable murine model for triple negative breast cancer and, in its early proliferative stage, is believed to be heavily infiltrated by immune cells, particularly active neutrophils and myeloid derived suppressor cells (MDSCs; Wang et al., 2017 and Bunt et al., 2006). The 4T1 tumor was clearly visible 1 h after injection of the radiopharmaceutical (FIG. 9), consistent with tumor inflammation. More experiments may be needed to prove actual uptake mediated by tumor infiltrated immune cells.

[¹⁸F]4FN PET was employed to image a model of inflammatory arthritis and validate its correlation with L012, a known ROS/RNS bioluminescence sensor. The PET tracer was able to readily image the inflammatory ROS burst, correlated strongly with L012, and was co-validated to concur with swelling and inflammatory infiltration (FIG. 10).

Finally, the biochemical dependence of [¹⁸F]4FN cellular retention on ROS/RNS was demonstrated in neutrophil-like human HL-60 cells, an all-trans-retinoic acid-differentiated model of neutrophils. There was a significant increase in [¹⁸F]4FN retention in activated cells vs vehicle (ratio>>1), that could be blocked by known inhibitors of Nox2 (DPI) and MPO (4-ABAH).

In addition, [¹⁸F]L012 was radiolabeled using the microfluidics automated module Advion NanoTek from commercially available L012 (Scheme 4, conditions under optimization). Identity of the PET tracer was proved by co-elution with the cold standard, the latter synthesized by reacting L012 with CsF.

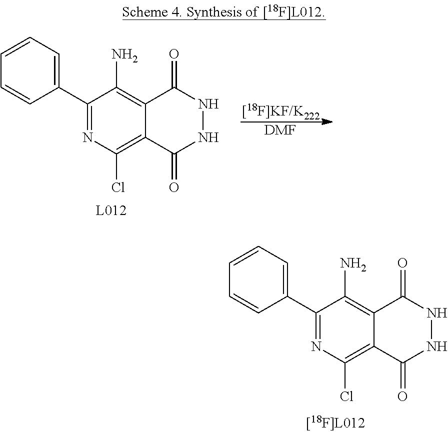

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the disclosure. All variations and modifications apparent to those skilled in the art are deemed to be within the spirit, scope, and concept of the disclosure as defined by the appended claims.

### VII. REFERENCES

The following references to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J, 22(3):659-661, 2008.
Smith, March's Advanced Organic Chemistry: Reactions, Mechanisms, and Structure, 7^{th} Ed., Wiley, 2013.
Aarts and Kuijpers, Eur J Clin Invest., 48(Suppl. 2):e12989, 2018.
Abe et al., J. Cereb. Blood Flow Metab. 34(12):1907-1913, 2014.
Acharya et al., J. Biol. Chem., 289(25):17406-17415, 2014.
Anastasi et al., Clin. Chem. Lab. Med., 56:1413, 2018.
Bilusic and Gulley, J Nat'l Cancer Inst, 109(12):djx132, 2017.
Breckwoldt et al., Proc Natl Acad Sci USA, 105(47):18584-18589, 2008.
Bunt et al., J Immunol, 176(1):284-290, 2006.
Chapman et al., J Biol Chem., 288(9):6465-6477, 2013.
Chen et al., Arch Med Res., 42:368-374, 2011.
Chen et al., Magn Reson Med, 52(5):1021-1028, 2004.
Corrêa et al., Front. Immunol., 8(1129), 2017.
Daiber et al., Free Radic Biol Med., 36:(1):101-111, 2004.
Frei et al., Proc Natl Acad Sci USA, 86(16):6377-6381, 1989.
Go et al., Circulation, 127 (1), 2013.
Goetzl, Am J Pathol., 85(2):419-436, 1976.
Goiffon et al., Nature Commun, 6:6271, 2015.
Gross et al., Nat. Med., 15:455-461, 2009.
Hager et al., J Intern Med., 268(1):25-34, 2010.
Hansson, N Engl J Med., 352(16):1685-1695, 2005.
Henson, J Immunol, 107(6):1535-1546, 1971.
Hou et al., ACS Chem. Neurosci., 9(3):578-586, 2018.
Jiemy et al., Autoimmun. Rev., 17(7):715-726, 2018.
Khan et al., Med. Sci., 6(2):33, 2018.
Kielland et al., Free Radic Biol Med., 47(6):760-766, 2009.
Klebanoff et al., J Leukoc Biol., 93(2):185-198, 2013.
Leliefeld et al., Front Immunol, 6:(471), 2015.
Li et al., J. Autoimmun., 2018.
Mayadas et al., Trends Mol Med., 16(8):368-378, 2010.
McDonnell et al., Clin Biochem., 42:549-561, 2009.
Morell et al., Clin Rev Allergy Immunol., 53(2):198-218, 2017.
Naghavi et al., Circulation, 108(15):1664-1672, 2003.
Nanni et al., J. Nucl. Med., 51(12):1932-1936, 2010.
Narukawa et al., Drug Metab Dispos., 32(7):758-761, 2004.
Nicholls and Hazen, Arteriosclerosis, thrombosis, and vascular biology, 25(6):1102-1111, 2005.
Owusu-Ansah et al., Protoc. Exch., doi:10. 1038/nprot.2008.23, 2008.
Park et al., Intest Res, 16(3):338-345, 2018.
Pierson et al., Clin. Immunol., 189:23-28, 2018.
Rakic et al., Cell Tissue Res., 371(3):517-529, 2018.
Rodriguez et al., J Am Chem Soc, 132(1):168-177, 2010.
Rymaszewski et al., Cancers, 6(2):1111, 2014.
Sakamoto et al., Clin Biochem., 41:584-590, 2008.
Schindhelm et al., Clin. Chem., 55(8):1462-1470, 2009.
Schultz and Kaminker, Arch Biochem Biophys., 96:465-467, 1962.
Sfanos et al., Nat. Rev. Urol., 15:11, 2017.
Signore et al., Semin Nucl Med., 47(3):286-303, 2017.
Skopelja-Gardner et al., J. Autoimmun., 88:1-10, 2018.
Soehnlein, Circ Res., 110(6):875-888, 2012.
Stone et al., EBioMedicine, 30:14-28, 2018.
Szmigielski and Michalewska, Folia Haematol Int Mag Klin Morphol Blutforsch., 101(4):536-542, 1974.
Terriere et al., Biochem J., 79(3):620-623, 1961.
Tredwell et al., Angew. Chem. Int. Ed., 53(30):7751-7755, 2014.
Violi et al., 2017
Wagner et al., Prev. Med., 54:S38-S41, 2012.
Wang et al., Cancer Immunol Immunother., 66(3):355-366, 2017.
Wojkowska et al., Mediators Inflamm, 2014:8, 2014.
Wu et al., Theranostics, 3(7):448-466, 2013.
Yin and Heit, Cell Tissue Res., 371(3):455-471, 2018.
Zhang et al., Molecules, 21(12):1696, 2016.
Zanzonico, Radiat. Dosimet., 41(2):297-308, 2000.
Zinnhardt et al., Theranostics, 8(10):2603-2620, 2018.

What is claimed is:

1. A radiolabeled compound of the formula: 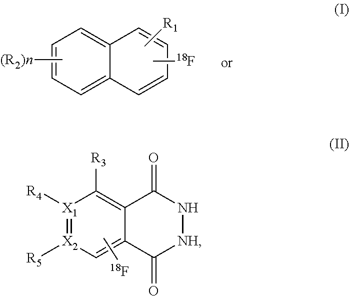
wherein:
n is 0-6;
R₁ is —ORₐ or —NR_{b}R_{c}, wherein:
Rₐ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{b} and R_{c} are each independently hydrogen or a monovalent amine protecting group; or
alkyl_{(C≤12)}, aralkyl_{(C≤12)}, acyl_{(C≤12)}, or a substituted version of any of these groups; or
R_{b} and R_{c} are taken together and are a divalent amine protecting group;
R₂, in each instance, is independently hydrogen, hydroxy, halo, amino, nitro, carboxy, or mercapto; or
—Y—R_{d}, wherein:
Y is a covalent bond, —C(O)—, —OC(O)—, —NHC(O)—;
R_{d} is alkyl_{(C≤12)}, alkoxy_{(C≤12)}, alkylamino_{(C≤12)}, dialkylamino_{(C≤12)}, or a substituted version of any of these groups;
R₃ is hydrogen, —ORₑ or —NR_{f}R_{g}, wherein:
Rₑ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{f} and R_{g} are each independently hydrogen or a monovalent amine protecting group; or
alkyl_{(C≤12)}, aralkyl_{(C≤12)}, acyl_{(C≤12)}, or a substituted version of any of these groups; or
R_{f} and R_{g} are taken together and are a divalent amine protecting group;
R₄ and R₅ are each independently absent, hydrogen, hydroxy, amino, cyano, nitro, or halo; or
alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
X₁ and X₂ are each independently —C═ or —N═;
or a pharmaceutically acceptable salt of either of these formulae.

2. The radiolabeled compound of claim 1, wherein the compound is further defined as: 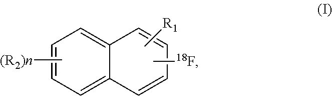
wherein:
n is 0-6;
R₁ is —ORₐ or —NR_{b}R_{c}, wherein:
Rₐ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{b} and R_{c} are each independently hydrogen or a monovalent amine protecting group; or
alkyl_{(C≤12)}, aralkyl_{(C≤12)}, acyl_{(C≤12)}, or a substituted version of any of these groups; or
R_{b} and R_{c} are taken together and are a divalent amine protecting group;
R₂, in each instance, is independently hydrogen, hydroxy, halo, amino, nitro, carboxy, or mercapto; or
—Y—R_{d}, wherein:
Y is a covalent bond, —C(O)—, —OC(O)—, —NHC(O)—;
R_{d} is alkyl_{(C≤12)}, alkoxy_{(C≤12)}, alkylamino_{(C≤12)}, dialkylamino_{(C≤12)}, or a substituted version of any of these groups;
or a pharmaceutically acceptable salt thereof.

3. The radiolabeled compound of either claim 1 or claim 2, wherein the compound is further defined as: 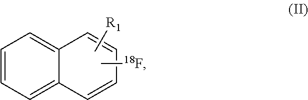
wherein:
R₁ is —ORₐ or —NR_{b}R_{c}, wherein:
Rₐ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{b} and R_{c} are each independently hydrogen or a monovalent amine protecting group; or
alkyl_{(C≤12)}, aralkyl_{(C≤12)}, acyl_{(C≤12)}, or a substituted version of any of these groups; or
R_{b} and R_{c} are taken together and are a divalent amine protecting group;
or a pharmaceutically acceptable salt thereof.

4. The radiolabeled compound according to any one of claims 1-3, wherein the compound is further defined as: 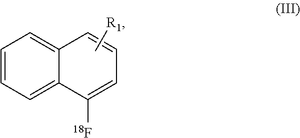
wherein:
R₁ is —ORₐ or —NR_{b}R_{c}, wherein:
Rₐ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{b} and R_{c} are each independently hydrogen or a monovalent amine protecting group; or
alkyl_{(C≤12)}, aralkyl_{(C≤12)}, acyl_{(C≤12)}, or a substituted version of any of these groups; or
R_{b} and R_{c} are taken together and are a divalent amine protecting group;
or a pharmaceutically acceptable salt thereof.

5. The radiolabeled compound according to any one of claims 1-4, wherein the compound is further defined as: 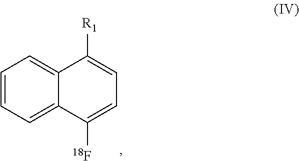
wherein:
R₁ is —ORₐ or —NR_{b}R_{c}, wherein:
Rₐ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{b} and R_{c} are each independently hydrogen or a monovalent amine protecting group; or
alkyl_{(C≤12)}, aralkyl_{(C≤12)}, acyl_{(C≤12)}, or a substituted version of any of these groups; or
R_{b} and R_{c} are taken together and are a divalent amine protecting group;
or a pharmaceutically acceptable salt thereof.

6. The radiolabeled compound according to any one of claims 1-5, wherein Rₐ is hydrogen, —S(O)₂OH, —C(O)-alkoxy_{(C≤12)}, substituted —C(O)-alkoxy_{(C≤12)}, heterocycloalkyl_{(C≤12)}, or substituted heterocycloalkyl_{(C≤12)}.

7. The radiolabeled compound according to any one of claims 1-6, wherein Rₐ is hydrogen.

8. The radiolabeled compound according to any one of claims 1-6, wherein Rₐ is —S(O)₂OH.

9. The radiolabeled compound according to any one of claims 1-6, wherein Rₐ is —C(O)-alkoxy_{(C≤12)} or substituted —C(O)-alkoxy_{(C≤12)}.

10. The radiolabeled compound according to any one of claims 1-6 and 9, wherein Rₐ is —C(O)-alkoxy_{(C≤12)}.

11. The radiolabeled compound according to any one of claims 1-6, 9, and 10, wherein Rₐ is —C(O)—OtBu.

12. The radiolabeled compound according to any one of claims 1-6, wherein Rₐ is heterocycloalkyl_{(C≤12)} or substituted heterocycloalkyl_{(C≤12)}.

13. The radiolabeled compound according to any one of claims 1-6 and 12, wherein Rₐ is substituted heterocycloalkyl_{(C≤12)}.

14. The radiolabeled compound according to any one of claims 1-6, 12, and 13, wherein Rₐ is 2-carboxy-3,4,5-trihydroxytetrahydro-2H-pyran-6-yl.

15. The radiolabeled compound of claim 1, wherein the compound is further defined as: 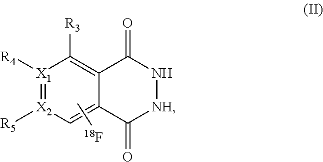
R₃ is hydrogen, —ORₑ or —NR_{f}R_{g}, wherein:
Rₑ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{f} and R_{g} are each independently hydrogen or a monovalent amine protecting group; or
alkyl_{(C≤12)}, aralkyl_{(C≤12)}, acyl_{(C≤12)}, or a substituted version of any of these groups; or
R_{f} and R_{g} are taken together and are a divalent amine protecting group;
R₄ and R₅ are each independently absent, hydrogen, hydroxy, amino, cyano, nitro, or halo; or
alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
X₁ and X₂ are each independently —C═ or —N═;
or a pharmaceutically acceptable salt thereof.

16. The radiolabeled compound of either claim 1 or claim 15, wherein the compound is further defined as: 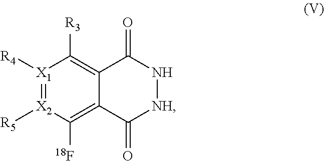
wherein:
R₃ is hydrogen, —ORₑ or —NR_{f}R_{g}, wherein:
Rₑ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{f} and R_{g} are each independently hydrogen or a monovalent amine protecting group; or
alkyl_{(C≤12)}, aralkyl_{(C≤12)}, acyl_{(C≤12)}, or a substituted version of any of these groups; or
R_{f} and R_{g} are taken together and are a divalent amine protecting group;
R₄ and R₅ are each independently absent, hydrogen, hydroxy, amino, cyano, nitro, or halo; or
alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
X₁ and X₂ are each independently —C═ or —N═;
or a pharmaceutically acceptable salt thereof.

17. The radiolabeled compound according to any one of claims 1, 15, and 16, wherein the compound is further defined as: 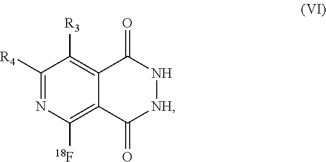
wherein:
R₃ is hydrogen, —ORₑ or —NR_{f}R_{g}, wherein:
Rₑ is hydrogen, —S(O)₂OH, or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
R_{f} and R_{g} are each independently hydrogen or a monovalent amine protecting group; or
alkyl_{(C≤12)}, aralkyl_{(C≤12)}, acyl_{(C≤12)}, or a substituted version of any of these groups; or
R_{f} and R_{g} are taken together and are a divalent amine protecting group;
R₄ is hydrogen, hydroxy, amino, cyano, nitro, or halo; or
alkyl_{(C≤12)}, aryl_{(C≤12)}, heteroaryl_{(C≤12)}, acyl_{(C≤12)}, alkoxy_{(C≤12)}, alkylamino_{(C≤12)}, dialkylamino_{(C≤12)}, or a substituted version of any of these groups; and
or a pharmaceutically acceptable salt thereof.

18. The radiolabeled compound according to any one of claims 1 and 15-17, wherein R₄ is aryl_{(C≤12)} or substituted aryl_{(C≤12)}.

19. The radiolabeled compound according to any one of claims 1 and 15-18, wherein R₄ is aryl_{(C≤12)}.

20. The radiolabeled compound according to any one of claims 1 and 15-19, wherein R₄ is phenyl.

21. The radiolabeled compound according to any one of claims 1 and 15-20, wherein R_{f} is hydrogen.

22. The radiolabeled compound according to any one of claims 1 and 15-21, wherein R_{g} is hydrogen.

23. The radiolabeled compound according to any one of claims 1-22, wherein the compound is further defined as: 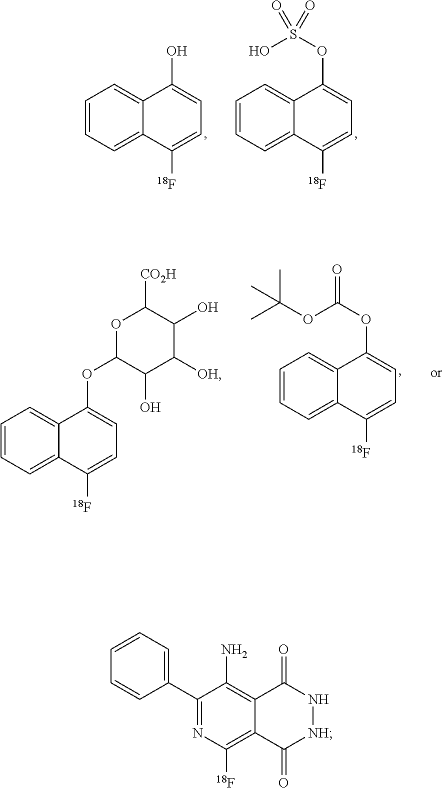
or a pharmaceutically acceptable salt thereof.

24. The radiolabeled compound according to any one of claims 1-23, wherein the compound is further defined as: 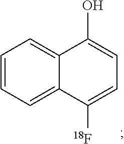 [SMILES: Oc1ccc([18F])c2ccccc12]
or a pharmaceutically acceptable salt thereof.

25. A radiopharmaceutical composition comprising:
(a) a radiolabeled compound according to any one of claims 1-24; and
(b) a pharmaceutically acceptable carrier.

26. The radiopharmaceutical composition of claim 25, wherein the composition is formulated for administration: intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, parenterally, subconjunctival, subcutaneously, via injection, via local delivery, or via localized perfusion.

27. The radiopharmaceutical composition of either claim 25 or claim 26, wherein the composition is formulated for administration intravenously or via injection.

28. The radiopharmaceutical composition according to any one of claims 25-27, wherein the composition is formulated for intravenous administration.

29. The radiopharmaceutical composition according to any one of claims 25-28, wherein the composition is formulated as a unit dose.

30. A method of imaging a subject comprising:
(a) administering to the subject an effective amount of a radiolabeled compound or radiopharmaceutical composition according to any one of claims 1-29; and
(b) obtaining at least one image of a portion of the subject.

31. The method of claim 30, wherein the subject is a vertebrate.

32. The method or claim 31, wherein the vertebrate is a mammal.

33. The method of claim 32, wherein the mammal is a human.

34. The method according to any one of claims 30-33, wherein the at least one image is a positron-emission tomography image.

35. The method according to any one of claims 30-34, wherein the method is suitable for detecting and/or measuring one or more biomarkers associated with inflammation.

36. The method according to any one of claims 30-34, wherein the method is suitable for detecting and/or measuring the activation of a biochemical pathway associated with inflammation.

37. The method of either claim 35 or claim 36, wherein the inflammation is caused by or results in an ROS and/or an RNS.

38. The method of claim 37, wherein the ROS and/or the RNS are produced by a Fenton reaction.

39. The method according to any one of claims 30-38, wherein the method further comprises detecting a level of activity of an enzyme.

40. The method of claim 39, wherein the enzyme is a peroxidase.

41. The method of either claim 39 or claim 40, wherein the enzyme is myeloperoxidase.

42. The method of claim 39, wherein the enzyme is an NADPH oxidase.

43. The method of claim 39 or claim 42, wherein the enzyme is NOX1, NOX2, NOX3, or NOX4.

44. The method according to any one of claims 39, 42, and 43, wherein the enzyme is NOX2.

45. The method of claim 39, wherein the enzyme is a nitric oxide synthase.

46. The method of either claim 39 or claim 45, wherein the enzyme is iNOS, nNOS, or eNOS.

47. The method according to any one of claims 39, 45, and 46, wherein the enzyme is iNOS.

48. The method of claim 39, wherein the enzyme is a xanthine oxidase or dual oxidase.

49. The method according to any one of claims 30-48, wherein the method further comprises diagnosing, prognosing, staging, or monitoring the progression of a disease or disorder.

50. The method of claim 49, wherein the disease or disorder is a cardiovascular disease, cancer, a neurological disorder, an autoimmune disease, obesity, a condition associated with radiation, a bacterial infection, a viral infection, a parasitic infection, or a condition associated with obesity, inflammation or a condition associated with inflammation.

51. The method of either claim 49 or claim 50, wherein the disease or disorder is obesity or a condition associated with obesity.

52. The method of either claim 49 or claim 50, wherein the disease or disorder is inflammation or a condition associated with inflammation.

53. The method of claim 52, wherein the disease or disorder is pancreatitis, hepatitis, pneumonitis, adult respiratory distress syndrome, pulmonary fibrosis, cystic fibrosis, chronic obstructive pulmonary disease, asthma, dermatitis, gastritis, esophagitis, encephalitis, dementias, irritable bowel syndrome, inflammatory bowel disease, nephritis, muscle wasting, osteoarthritis, type 2 diabetes or a complication of type 1 or type 2 diabetes.

54. The method of claim 53, wherein the disease or disorder is pneumonitis or nephritis.

55. The method of claim 53, wherein the disease or disorder is type 2 diabetes or a complication of type 1 or type 2 diabetes.

56. The method of claim 50, wherein the disease or disorder is a neurological disorder.

57. The method of claim 56, wherein the neurological disorder is a central neurologic disease.

58. The method of claim 57, wherein the central neurological disease is white matter inflammation, meningitis, vasculitis, autoimmune encephalitis, metabolic encephalitis, Alzheimer's Disease, dementias, or degenerative inflammatory diseases of the brain.

59. The method of claim 50, wherein the disease or disorder is a condition associated with radiation.

60. The method of claim 59, wherein the disease or disorder is post-radiation inflammation or fibrosis.

61. The method of claim 50, wherein the disease or disorder is a cardiovascular disease.

62. The method of claim 61, wherein the cardiovascular disease is vasculitis, atherosclerosis, myocardial infarction, myocarditis, heart failure, pulmonary hypertension, or stroke.

63. The method of claim 62, wherein the cardiovascular disease is atherosclerosis.

64. The method according of claim 50, wherein the disease or disorder is cancer.

65. The method according of claim 64, wherein the cancer is breast cancer, liver cancer, lung cancer, thyroid cancer, head and neck cancer, pancreatic cancer, colorectal cancer, prostate cancer, renal cancer, skin cancer, brain cancer, sarcoma, multiple myeloma, lymphoma, or leukemia.

66. The method according of claim 65, wherein the cancer is breast cancer.

67. The method of either claim 65 or 66, wherein the breast cancer is inflammatory breast cancer.

68. The method according to any one of claims 65-67, wherein the breast cancer is triple-negative breast cancer.

69. The method according of claim 65, wherein the cancer is skin cancer.

70. The method according of claim 69, wherein the skin cancer is melanoma.

71. The method according of claim 65, wherein the cancer is brain cancer.

72. The method according of claim 71, wherein the brain cancer is glioblastoma.

73. The method according of claim 50, wherein the disease or disorder is an autoimmune disease.

74. The method according of claim 73, wherein the autoimmune disease is psoriasis, multiple sclerosis, scleroderma, rheumatoid arthritis, lupus, psoriatic arthritis, ankylosing spondylitis, Sjögren syndrome, vitiligo, uveitis, dry eye syndrome, systemic sclerosis, type 1 diabetes, encephalitis, myasthenia gravis, or inflammatory bowel disease.

75. The method of claim 50, wherein the disease or disorder is a viral infection, a bacterial infection, or a parasitic infection.

76. The method of either claim 50 or claim 52, wherein the disease or disorder is inflammation associated with a vector-borne disease.

77. The method according of claim 74, wherein the autoimmune disease is multiple sclerosis.

78. The method of according to any one of claims 30-77, wherein the administering is via injection.

79. The method according to any one of claims 30-78, wherein the method further comprises monitoring the progression of tissue repair.

80. A precursor compound of the formula: 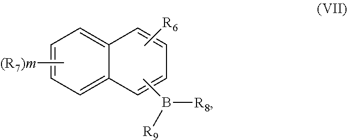
wherein:
m is 0-6;
R₆ is —ORₕ or —NRᵢRⱼ, wherein:
Rₕ is —S(O)₂OH or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
Rᵢ and Rⱼ are each independently hydrogen or a monovalent amine protecting group; or
alkyl_{(C≤12)}, aralkyl_{(C≤12)}, acyl_{(C≤12)}, or a substituted version of any of these groups; or
Rᵢ and Rⱼ are taken together and are a divalent amine protecting group;
R₇, in each instance, is independently hydroxy, halo, amino, nitro, carboxy, or mercapto; or
—Y—Rₖ, wherein:
Y is a covalent bond, —C(O)—, —OC(O)—, —NHC(O)—;
Rₖ is alkyl_{(C≤12)}, alkoxy_{(C≤12)}, alkylamino_{(C≤12)}, dialkylamino_{(C≤12)}, or a substituted version of any of these groups; and
R₈ and R₉ are each independently halo or hydroxy; or
alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
X₃ is alkanediyl_{(C≤12)}, substituted alkanediyl_{(C≤12)}, or a boronic acid protecting group;
or a pharmaceutically acceptable salt thereof.

81. The precursor compound of claim 80, wherein the compound is further defined as: 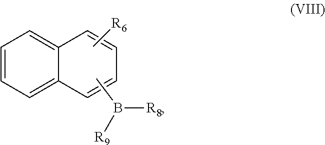
wherein:
R₆ is —ORₕ or —NRᵢRⱼ, wherein:
Rₕ is —S(O)₂OH or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
Rᵢ and Rⱼ are each independently hydrogen or a monovalent amine protecting group; or
alkyl_{(C≤12)}, aralkyl_{(C≤12)}, acyl_{(C≤12)}, or a substituted version of any of these groups; or
Rᵢ and Rⱼ are taken together and are a divalent amine protecting group; and
R₈ and R₉ are each independently halo or hydroxy; or
alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
X₃ is alkanediyl_{(C≤12)}, substituted alkanediyl_{(C≤12)}, or a boronic acid protecting group;
or a pharmaceutically acceptable salt thereof.

82. The precursor compound of either claim 80 or claim 81, wherein the compound is further defined as: 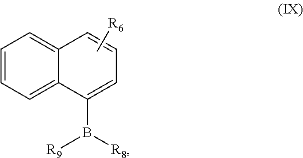
wherein:
R₆ is —ORₕ or —NRᵢRⱼ, wherein:
Rₕ is —S(O)₂OH or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
Rᵢ and Rⱼ are each independently hydrogen or a monovalent amine protecting group; or
alkyl_{(C≤12)}, aralkyl_{(C≤12)}, acyl_{(C≤12)}, or a substituted version of any of these groups; or
Rᵢ and Rⱼ are taken together and are a divalent amine protecting group; and
R₈ and R₉ are each independently halo or hydroxy; or
alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
X₃ is alkanediyl_{(C≤12)}, substituted alkanediyl_{(C≤12)}, or a boronic acid protecting group;
or a pharmaceutically acceptable salt thereof.

83. The precursor compound according to any one of claims 80-82, wherein the compound is further defined as: 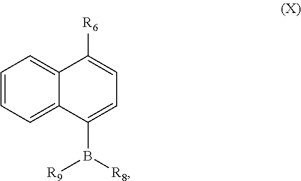
wherein:
R₆ is —ORₕ or —NRᵢRⱼ, wherein:
Rₕ is —S(O)₂OH or a hydroxyl protecting group; or
alkyl_{(C≤12)}, cycloalkyl_{(C≤12)}, heterocycloalkyl_{(C≤12)}, aralkyl_{(C≤12)}, acyl_{(C≤12)}, —C(O)-alkoxy_{(C≤12)}, alkylsulfonyl_{(C≤12)}, alkoxysulfonyl_{(C≤12)}, alkylaminosulfonyl_{(C≤12)}, dialkylaminosulfonyl_{(C≤12)}, or a substituted version of any of these groups;
Rᵢ and Rⱼ are each independently hydrogen or a monovalent amine protecting group; or
alkyl_{(C≤12)}, aralkyl_{(C≤12)}, acyl_{(C≤12)}, or a substituted version of any of these groups; or
Rᵢ and Rⱼ are taken together and are a divalent amine protecting group; and
R₈ and R₉ are each independently halo or hydroxy; or
alkoxy_{(C≤12)}, substituted alkoxy_{(C≤12)}, acyloxy_{(C≤12)}, or substituted acyloxy_{(C≤12)}; or
R₈ and R₉ are taken together and is —O—X₃—O—, wherein:
X₃ is alkanediyl_{(C≤12)}, substituted alkanediyl_{(C≤12)}, or a boronic acid protecting group;
or a pharmaceutically acceptable salt thereof.

84. The precursor compound according to any one of claims 80-83, wherein Rₕ is a hydroxyl protecting group.

85. The precursor compound according to any one of claims 80-84, wherein Rₕ is heterocycloalkyl_{(C≤12)} or substituted heterocycloalkyl_{(C≤12)}.

86. The precursor compound according to any one of claims 80-85, wherein Rₕ is substituted heterocycloalkyl_{(C≤12)}.

87. The precursor compound according to any one of claims 80-86, wherein Rₕ is 2-carboxy-3,4,5-trihydroxytetrahydro-2H-pyran-6-yl.

88. The precursor compound according to any one of claims 80-84, wherein Rₕ is —C(O)-alkoxy_{(C≤12)}.

89. The precursor compound according to any one of claims 80-84 and 88, wherein Rₕ is —C(O)—OtBu.

90. The precursor compound according to any one of claims 80-89, wherein R₈ is hydroxy.

91. The precursor compound according to any one of claims 80-89, wherein R₈ is alkoxy_{(C≤12)}.

92. The precursor compound according to any one of claims 80-91, wherein R₉ is hydroxy.

93. The precursor compound according to any one of claims 80-91, wherein R₉ is alkoxy_{(C≤12)}.

94. The precursor compound according to any one of claims 80-89, wherein R₈ and R₉ are taken together and are alkanediyl_{(C≤12)}.

95. The precursor compound according to any one of claims 80-89 and 94, wherein R₈ and R₉ are taken together and are 1,1,2,2-tetramethylethanediyl.

96. The precursor compound according to any one of claims 80-95, wherein the compound is further defined as: 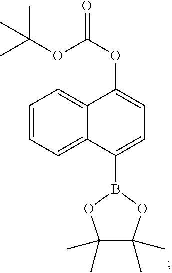 [SMILES: CC(C)(C)OC(=O)Oc1ccc(B2OC(C)(C)C(C)(C)O2)c2ccccc12]
or a pharmaceutically acceptable salt thereof.

97. A precursor composition comprising:
(a) a precursor compound according to any one of claims 80-96; and
(b) a pharmaceutically acceptable carrier.

98. A radiopharmaceutical kit for the preparation of a radiolabeled compound according to any one of claims 1-29, wherein the kit comprises a precursor compound or composition according to any one of claims 80-97.

99. The radiopharmaceutical kit of claim 98, wherein the kit comprises or consists of a cassette.

100. The radiopharmaceutical kit of claim 98, wherein the kit further comprises a pharmaceutically acceptable carrier.

101. The radiopharmaceutical kit of either claim 98 or claim 100, wherein the kit further comprises instructions for the preparation of the radiolabeled compound.
